(19) United States
(12) Patent Application Publication
(10) Pub. No.: US 2008/0172072 A1
(43) Pub. Date: Jul. 17, 2008

(54) Internal sensors for use with gastric restriction devices

(75) Inventors: Scott Pool, Laguna Hills, CA (US); Jay R. McCoy, Temecula, CA (US); Richard L. Quick, Mission Viejo, CA (US); Blair Walker, Mission Viejo, CA (US); Shahram Moaddeb, Irvine, CA (US); Arvin Chang, West Covina, CA (US)

Correspondence Address: Vista IP Law Group LLP, 2040 MAIN STREET, 9TH FLOOR, IRVINE, CA, 92614, US

(73) Assignee: Ellipse Technologies, Inc., Irvine, CA (US)

(21) Appl. No.: 11/888,537

(22) Filed: Jul. 31, 2007

Related U.S. Application Data
(60) Provisional application No. 60/880,080, filed on Jan. 11, 2007.
(60) Provisional application No. 60/904,625, filed on Mar. 1, 2007.

(51) Int. Cl. A61B 17/08

(52) U.S. Cl. 606/151

(57) ABSTRACT

Apparatus and methods of monitoring gastric restriction devices are described. Internally mounted sensors detect at least one of a quantity of a test substance, a flow through the stomal opening produced by a restriction device, slippage of the device, and erosion of the gastric wall. In some embodiments flow versus no flow can be determined, or a flow rate can be calculated. Monitoring of internally mounted sensors permits optimization of the performance of a gastric restriction device, using noninvasive techniques.

### RELATED APPLICATIONS

This application claims the benefit under 35 U.S.C. § 119(e) of U.S. Provisional Application No. 60/853,105, filed Oct. 20, 2006, and entitled “GASTROINTESTINAL RESTRICTION DEVICE”; U.S. Provisional Application No. 60/854,574, filed Oct. 25, 2006, and entitled “GASTROINTESTINAL RESTRICTION DEVICE”; U.S. Provisional Application No. 60/880,080, filed Jan. 11, 2007, and entitled “SENSORS FOR USE WITH GASTRIC RESTRICTION DEVICE”; and U.S. Provisional Application, 60/904,625, filed Mar. 1, 2007, and entitled “NONINVASIVE METHODS AND APPARATUS FOR MONITORING AND ADJUSTING GASTRIC BANDS,” the entirety of all of which are hereby incorporated by reference.

### FIELD OF THE INVENTIONS

The present disclosure relates to apparatus and methods for monitoring and regulating gastric restriction devices. In particular, some embodiments are directed to internally located sensors for detecting flow or for determining flow rate through such a device, as well as to detecting slippage of the device or erosion of the gastric wall.

### BACKGROUND

At present, obesity is an ever-increasing public health problem not only in the United States but in a number of other countries. In the United States, it is estimated that more than 55%, or nearly 100 million adults, are overweight. Obesity can range from overweight to obese, and even super-obese. The degree of obesity is typically characterized using a measure known as body-mass-index, or BMI. The BMI takes into account the individual's height and weight in order to establish a relative index of obesity (See Table 1).

It is well-established in the medical literature that obesity adversely affects general health, and can result in reduced quality of life and reduced lifespan. It is now well-accepted that obesity is associated with increased risk of cardiovascular disease, diabetes and other health issues. In contrast, animal studies suggest that longevity is increased in lean subjects (See, e.g., Weindruch, R. & Walford, R. L., 1988, The Retardation of Aging and Disease by Dietary Restriction, Thomas, Springfield, Ill.; Spindler, S. R., 2003, in Anti-Aging Therapy for Plastic Surgery, eds. Kinney, B. & Carraway, J., Quality Medical, St. Louis, Mo.).

TABLE 1
Risk of Associated Disease According to BMI and Waist Size

| | | Disease Risk | Disease Risk |
|---|---|---|---|
| | | Waist ≦40 | Waist >40 |
| | Weight | in. (men) or | in. (men) or |
| BMI | Classification | 35 in. (women) | 35 in. (women) |
| 18.5 or less | Underweight | — | N/A |
| 18.5 to 24.9 | Normal | — | N/A |
| 25.0 to 29.9 | Overweight | Increased | High |
| 30.0 to 34.9 | Obese Class I | High | Very High |
| 35.0 to 39.9 | Obese Class 2 | Very High | Very High |
| 40.0 to 49.9 | Morbidly Obese | Extremely High | Extremely High |
| >49.9 | Super Obese | Extremely High | Extremely High |

A number of approaches have been developed to deal with obesity as a means to improving individual health. The simplest method, dieting, can be effective but only if the individual adheres to a program of caloric restriction and exercise. Thus, even though dieting is relatively popular, many persons have difficulty in maintaining the long-term discipline needed for dieting to be an effective weight loss and weight maintenance regime.

As a result, medical methods have been developed in order to assist people in losing weight and maintaining weight within normal ranges. Bariatrics is the branch of medicine concerned with the management of obesity and associated diseases. Several surgical methods have been developed that seek to effectively reduce caloric intake. These include procedures such as gastric bypass, gastroplasty, also known as stomach stapling and adjustable gastric banding.

In gastric bypass, a surgeon permanently changes the shape of the stomach by surgical reduction in order to create a smaller gastric pouch, or “new stomach.” The remainder of the stomach is then divided and separated from this pouch, thus reducing the amount of food that can be ingested. In addition, it is typical to bypass a portion of the small intestine, further reducing caloric uptake by reducing absorption in the gut. Once complete, this form of surgery is effectively irreversible.

In gastroplasty, the surgeon staples the upper stomach to create a small pouch with a capacity of about 1-2 ounces. A small stomal opening or lumen connects the upper stomach pouch and the remainder of the stomach. No changes are made to the remainder of the digestive tract, and so this method is purely restrictive in nature.

A newer and relatively less invasive procedure involves the use of an adjustable band to provide essentially the same result as a gastroplastic procedure, without the need to open the gastric cavity or perform any cutting or stapling operations. These bands are variously referred to in the literature as adjustable gastric restriction devices, adjustable gastric bands, or simply gastric bands.

One such device is the Inamed LAP-BAND®. This device is essentially an annular balloon that is placed around a portion of the stomach, forming the stomach into upper and lower pouches with a stomal opening, or lumen, connecting the two regions. The balloon is then inflated, typically with a saline solution, progressively closing the annulus around the stomach and reducing the size of the lumen connecting the upper and lower portions of the stomach. The first adjustment is usually performed several weeks after surgical placement of the gastric band, in order to allow time for the patient to heal from the initial surgical procedure, and to allow a fibrous tissue capsule to form around the band. The band can be inflated or deflated as necessary to alter the size of the stoma, thus providing at least in theory a method to tailor the device to each individual.

### SUMMARY

However, despite the advantages provided by gastric banding techniques, a number of drawbacks remain. These include slippage, erosion, infection, patient discomfort, pain during the adjustment procedure, and an inability to determine the correct adjustment amount without using X-ray fluoroscopy in conjunction with ingesting a contrast solution to monitor flow past the restriction.

Slippage may occur if a gastric band is adjusted too tight, or too loose, depending on the situation and the type of slippage. Slippage can also occur in response to vomiting, as occurs when a patient eats more food that can be comfortably accommodated in the upper pouch. During slippage, the size of the upper pouch may grow, causing the patient to be able to consume a larger amount of food before feeling full, thus lowering the effectiveness of the gastric band.

On the other hand, erosion may occur if the gastric band is adjusted too tight, or if the band is sutured too tightly to the stomach wall. In either case, reducing the risk of slippage or erosion may be accomplished by adjusting the device to provide a proper flow rate. Often, erosion stems from an infection or a foreign body reaction.

With inflatable gastric bands, infection and patient discomfort can arise due to repeated use of a hypodermic needle in order to adjust the relative “inflation” of the band. Non-invasively adjustable gastric bands have been proposed, some of which permit adjustment of the band without the need for invasive techniques such as needles. These bands also seek to provide a correct reading of the inner diameter of the gastric band at all times. However, because the wall thickness of the stomach is not uniform from patient to patient, the actual inner diameter of the stoma produced by the gastric band is unknown. Thus, the size of the opening of the band is at best an approximation of the stomal opening that connects the smaller upper pouch and the remainder of the stomach, and not necessarily predictive of the performance to be expected from the gastric band.

In order to properly monitor movement of material through the stoma, methods that directly measure flow of material through the stomach are preferred. Presently, no method exists for easily determining flow through the stoma. Flow is typically monitored when the gastric band is adjusted, by tracking of a swallowed barium suspension, for example BAROSPERSE® or EZ-PAQUE®, and then visualizing the movement of the suspension by X-ray fluoroscopy.

However, the use of fluoroscopy presents its own problems, significantly increasing the exposure of the patient to X-rays. As X-rays are a form of ionizing radiation, their use should always be with deference to the additional risk that radiation poses to humans. In certain patients, the risk of radiation is increased. For example, a large percentage of patients receiving gastric bands are women in their child bearing years. The few first weeks of pregnancy, when a mother may be unaware she is pregnant, is an especially critical time of fetal development and exposure to X-rays is to be avoided if at all possible.

In addition, in many centers, the use of X-ray fluoroscopy is cost-prohibitive, and often, the patient either lacks insurance coverage, or otherwise is unable to afford this kind of follow-up treatment. As an alternative, many centers do not use barium in combination with X-ray fluoroscopy but rather have the patient simply drink a quantity of water. If the water does not pass, the gastric band is loosened. However, using this method, it is impossible to determine with any precision as to how tight or loose the band might be, other than in the most qualitative sense of whether there is an opening. In addition, even though water passes through the opening, the band may still be too tight to permit solid food to pass leading to patient discomfort and an increased risk of vomiting. The relatively high stresses imposed by vomiting increase the risk of movement or slippage of the band, in addition to increasing the patient's level of discomfort and anxiety.

The results will also vary depending on the patient's ability to sense movement of the ingested substance past the restriction. Some patients may be more aware of gastric sensations than others, and so a wide variability in adjustment would be expected from patient to patient, depending on their ability to accurately convey to the physician whether they believe material to be passing the restriction.

Another perplexing factor is the fact that sometimes gastric bands display a diurnal variation. For example, the device may be tighter in the morning and looser in the evening. When adjustments are performed, it is not possible to know beforehand whether an initial adjustment of the opening produced by the band will be effective. Consequently, depending upon what time of day the gastric band is placed and adjusted, varying results may be seen in terms of flow of contents past the restriction.

Serious complication can arise from improper adjustment. For example, if the stomal opening produced by a band that is initially adjusted and considered to be adjusted correctly subsequently becomes blocked, such that even water fails to pass, the patient is in danger of quickly becoming dehydrated, a dangerous situation that may require emergent care.

While the use of barium suspension allows for visualization of the movement of material through the stomal opening, and provides a quantifiable method of adjustment, barium suspensions as typically used (e.g., 66% barium sulphate by weight in water) are several hundred times more viscous than water. Barium suspensions also exhibit non-Newtonian flow properties, making movement characteristics more difficult to predict. Even at reduced amounts (e.g., 25% barium sulphate by weight in water), the solution is still 15 to 20 times as viscous as water. Where the gastric band produces a very small stomal opening, viscous solutions may fail to flow through the opening.

Different patients require different degrees of restriction depending on their eating habits, motivation, and other factors. Thus, at times it is desirable to adjust a gastric band to produce a very small stomal opening in order to achieve optimal weight control results. However, with very small openings, the viscosity of the barium suspension may not permit reliably detectable flow, and thus the restriction may be adjusted to provide a larger stoma than would be optimal in the particular case. It is also recognized that drinking barium suspensions is not pleasant to the patient due to the taste and texture of the material. Barium is also known to cause diarrhea in some individuals.

Alternative radio-opaque solutions are available that are iodine-based, for example Gastrografin. Gastrografin has a reported viscosity of 18.5 cP at 20° C. and 8.9 cP at 37° C. Consequently, as with barium suspensions, this is several times the viscosity of water, and in lower viscosity dilutions, the visibility using X-ray fluoroscopy is reduced. There is also an added risk in that some patients are allergic to iodine-based contrast agents such as Gastrografin, although this is typically only seen when the agent is administered intravenously. Thus, the use of all contrast solutions, whether barium-based, iodine-based or others, entails additional cost and risk.

Because of the present limitations in prior art methods for monitoring and adjusting gastric restriction devices such as gastric bands, it would be desirable to have a noninvasive method both for calibrating these devices, and later post-operative monitoring of their function, in order to provide patients with an optimal combination of weight loss benefit, along with reduced cost and risk to health.

Accordingly, in some embodiments, there is provided a system for use in controlling food intake in a patient, the system comprising: a gastric restriction device that engages the patient's stomach between proximal and distal gastric regions that are connected by a stomal opening; and a sensor coupled to the gastric restriction device; wherein the sensor is configured to reside within the patient's body when the gastric restriction device is engaged with the stomach; and wherein the sensor outputs information indicative of at least one of: a presence of a test substance within the stomach; a movement of the test substance within the stomach; a movement of the gastric restriction device from a first position to a second position; and an erosion of a wall of the stomach.

In some embodiments, the sensor outputs information indicative of a presence of a test substance within the stomach. In some embodiments, the sensor outputs information indicative of a movement of the test substance within the stomach. In some embodiments, the sensor outputs information indicative of a movement of the gastric restriction device from a first position to a second position. In some embodiments, the sensor outputs information indicative of and an erosion of a wall of the stomach.

In some embodiments, the information indicative of the movement of the test substance comprises at least one of a flow rate, a flow velocity, and an occurrence of flow.

In some embodiments, the sensor is electrically coupled to the gastric restriction device. In some embodiments, the sensor is mechanically coupled to the gastric restriction device. In some embodiments, the sensor is located in or on the gastric restriction device.

In some embodiments, the sensor comprises a thermal sensor that detects a temperature that is influenced by the test substance. In some embodiments, the sensor comprises a magnetic field sensor that detects a magnetically detectable property of the test substance. In some embodiments, the sensor comprises a radiofrequency receiver that detects an electromagnetic signal emitted by the test substance. In some embodiments, the sensor comprises a capacitance sensor. In some embodiments, the capacitance sensor detects a substance present in the stomal opening. In some embodiments, the sensor comprises an impedance sensor. In some embodiments, the impedance sensor detects an indication of the erosion of the wall of the stomach. In some embodiments, the sensor comprises a pH sensor. In some embodiments, the pH sensor detects at least one of a substance present in the stomal opening and an indication of the erosion of the wall of the stomach. In some embodiments, the sensor comprises an oxygen sensor, effective to detect an indication of the erosion of the wall of the stomach. In some embodiments, the sensor detects light. In some embodiments, the sensor detects acoustic energy. In some embodiments, the sensor is configured to detect Doppler shift echoes from ultrasound.

In some embodiments, the system further comprises the test substance. In some embodiments, the test substance comprises at least one of a food, a beverage, and a gastric secretion. In some embodiments, the test substance comprises a fluid. In some embodiments, the fluid has a viscosity between about 0.5 cP and about 2.0 cP at 20° C. In some embodiments, the test substance comprises a scattering agent that increases an echogenicity of the fluid. In some embodiments, the test substance emits or reflects acoustic energy. In some embodiments, the acoustic energy comprises at least one of audible sound, ultrasound, and Doppler shift echoes. In some embodiments, the test substance comprises an effervescent solution. In some embodiments, the test substance comprises an acoustic capsule. In some embodiments, the test substance comprises at least one of a light-reflecting material and a light-absorbing material.

In some embodiments, the system further comprises a telemetry unit that relays an output signal from a portion of the system that is inside the patient's body to an external receiver located outside the patient's body. In some embodiments, the telemetry unit further comprises a telemetry unit processor that processes a signal outputted from the sensor. In some embodiments, the system further comprises the external receiver. In some embodiments, the system further comprises a display, operative to indicate at least one of: the presence of the test substance within the stomach; the movement of the test substance within the stomach; the movement of the gastric restriction device from the first position to the second position; and the erosion of the wall of the stomach. In some embodiments, the display provides an alert that is at least one of audible, visible, and tactile. In some embodiments, the alert comprises at least one of an audible tone, an LED, a video display, a numerical display, vibration, and heat.

In some embodiments, there is provided a method, of monitoring performance or placement of a gastric restriction device, comprising: with a gastric restriction device, engaging a patient's stomach between proximal and distal gastric regions that are connected by a stomal opening; with a sensor that is coupled to the gastric restriction device and that resides within the patient's body when the gastric restriction device is engaged with the stomach, sensing information indicative at least one of: a presence of a test substance within the stomach; a movement of a test substance within the stomach; a movement of the gastric restriction device from a first position to a second position; and a presence of an erosion of a wall of the stomach.

In some embodiments, the method comprises sensing information indicative of a presence of a test substance within the stomach. In some embodiments, the method comprises sensing information indicative of a movement of a test substance within the stomach. In some embodiments, the method comprises sensing information indicative of a movement of the gastric restriction device from a first position to a second position. In some embodiments, the method comprises sensing information indicative of a presence of an erosion of a wall of the stomach. In some embodiments, the information indicative of the movement of a test substance comprises at least one of a flow rate, a flow velocity, and an occurrence of a flow.

In some embodiments, the method further comprises transmitting data based on the sensed information, located inside the patient's body, to an external receiver located outside of the patient's body. In some embodiments, the method further comprises adjusting a size of the gastric restriction device based on the transmitted data. In some embodiments, the method further comprises adjusting the gastric restriction device to achieve a flow rate of the test substance through the stomal opening in a range between about 1 mL per second and about 20 mL per second. In some embodiments, the method further comprises adjusting the gastric restriction device to achieve a flow rate of the test substance through the stomal opening in a range from about 5 mL per second to about 15 mL per second. In some embodiments, the test substance is equilibrated to a temperature about equal to a body temperature of the patient prior to administration of the test substance to the patient.

In some embodiments, the sensor is located in or on the gastric restriction device. In some embodiments, the sensor comprises a thermal sensor, and the test substance has a temperature distinguishable from a body temperature of the patient. In some embodiments, the sensor comprises a magnetic sensor, and the test substance has magnetically detectable properties. In some embodiments, the sensor comprises a radiofrequency receiver, and the test substance comprises a radio transmitter. In some embodiments, the sensor comprises a capacitance sensor. In some embodiments, the capacitance sensor detects a substance present in the stomal opening. In some embodiments, the sensor comprises an impedance sensor. In some embodiments, the impedance sensor detects an indication of the erosion of the wall of the stomach. In some embodiments, the sensor comprises a pH sensor. In some embodiments, the pH sensor detects at least one of a substance present in the stomal opening and an indication of the erosion of the wall of the stomach. In some embodiments, the sensor comprises an oxygen sensor, effective to detect an indication of the erosion of the wall of the stomach.

In some embodiments, the method further comprises administering the test substance to the patient. In some embodiments, the test substance comprises at least one of a food, a beverage, and a gastric secretion. In some embodiments, the test substance comprises a fluid. In some embodiments, the fluid has a viscosity between about 0.5 cP and about 2.0 cP at 20° C. In some embodiments, the test substance emits or reflects acoustic energy. In some embodiments, the acoustic energy comprises at least one of audible sound, ultrasound, and Doppler shift echoes. In some embodiments, the test substance comprises an effervescent solution. In some embodiments, the test substance comprises an acoustic capsule. In some embodiments, the method further comprises sensing acoustic energy with the sensor. In some embodiments, the method further comprises sensing light energy with the sensor. In some embodiments, the test substance comprises at least one of a light-reflecting material and a light-absorbing material. In some embodiments, the sensor is configured to detect Doppler shift echoes from ultrasound. In some embodiments, the test substance comprises a scattering agent that increases an echogenicity of the fluid.

In some embodiments there is provided a system for use in controlling food intake in a patient, the system comprising: means for engaging the patient's stomach between proximal and distal gastric regions that are connected by a stomal opening; and means for sensing coupled to the means for engaging; wherein the means for sensing is configured to reside within the patient's body when the means for engaging is engaged with the stomach; and wherein the means for sensing outputs information indicative of at least one of a presence of a test substance within the stomach, a movement of a test substance within the stomach, a movement of the gastric restriction device from a first position to a second position, and a presence of an erosion of a wall of the stomach. In some embodiments, the means for sensing output information indicative of a presence of a test substance within the stomach. In some embodiments, the means for sensing output information indicative of a movement of a test substance within the stomach. In some embodiments, the means for sensing output information indicative of a movement of the gastric restriction device from a first position to a second position. In some embodiments, the means for sensing output information indicative of a presence of an erosion of a wall of the stomach.

### BRIEF DESCRIPTION OF THE DRAWINGS

FIG. 1 illustrates a sectional view of the esophagus and stomach of a gastric restriction device patient undergoing a barium flow evaluation.

FIG. 2 illustrates a sectional view of the esophagus and stomach of a gastric restriction device patient undergoing a barium flow evaluation.

FIG. 3 illustrates a sectional view of the esophagus and stomach of a gastric restriction device patient where the stomal opening is closed.

FIG. 4 illustrates a sectional view of the esophagus and stomach of a gastric restriction device patient where the device has slipped from its initial placement location.

FIG. 5 illustrates a view of an embodiment for detecting a sound producing fluid.

FIG. 6 illustrates a section view of an embodiment where an acoustic capsule is used.

FIG. 7 illustrates a sectional view of an embodiment where an effervescent solution and inactivating solution are used.

FIG. 8A illustrates a gastric restriction device including an internally mounted ultrasound probe and detector combination.

FIG. 8B shows a schematic of the principles underlying measurement of fluid velocity by Doppler ultrasound.

FIG. 8C illustrates a gastric restriction device comprising a passive ultrasonic implant.

FIG. 8D illustrates a gastric restriction device comprising an angled Doppler transducer.

FIG. 9 is a sectional view of an embodiment where scattering agents are included in the test substance.

FIG. 10 is a sectional view of the stomach with a gastric restriction device comprising an integral sensor.

FIG. 11A illustrates an embodiment of a gastric restriction device comprising an integral sensor used to detect erosion of the stomach wall.

FIG. 11B illustrates a sectional view of the gastric restriction device of FIG. 11A.

FIG. 12A illustrates Doppler ultrasound recording data obtained from a patient.

FIG. 12B illustrates spectral analysis derived from the data presented in FIG. 12A.

FIG. 12C illustrates hypothetical data from an internally mounted thermal sensor.

FIG. 13 illustrates a side view of a slippage monitor.

FIG. 14 illustrates a block diagram showing relationships between a sensor, and associated telemetry and data handling components.

FIG. 15 illustrates a cross-section of a stoma compressed by a gastric restriction device comprising a capacitive sensor.

FIG. 16 illustrates a cross-section of a stoma compressed by a gastric restriction device comprising a capacitive sensor, wherein the stoma is more compressed than in FIG. 15.

FIG. 17 illustrates an embodiment, for sensing a magnetic or conductive fluid, and comprising an electromagnetic sensor.

FIGS. 18-19 illustrate some embodiments for sensing sound of a test substance using a sound pipe.

FIGS. 20-21 illustrate some embodiments for sensing temperature variation caused by a test substance.

FIGS. 22-23 illustrate some embodiments for sensing light correlated with the passage of a test substance.

### DETAILED DESCRIPTION

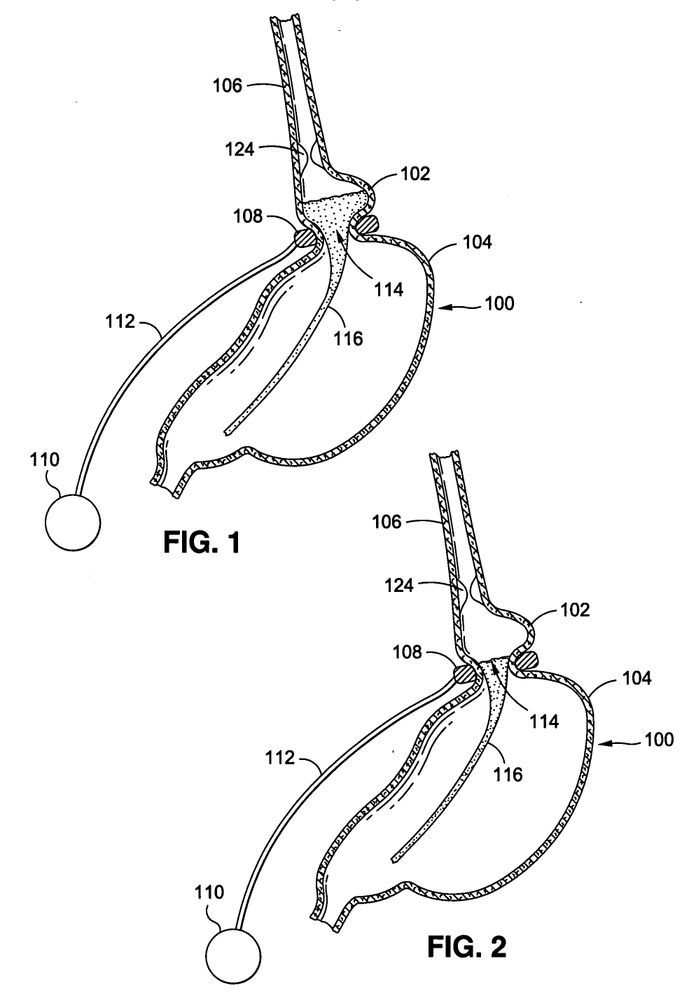

FIG. 1 illustrates a method of monitoring a gastric restriction device. A patient undergoes a visual flow rate evaluation test typically using barium contrast suspension 116 and X-ray fluoroscopy. The barium contrast solution 116 is radiopaque and is visualized using X-ray radiography. A gastric restriction device 108 is placed around the stomach 100, separating the stomach into an upper stomach pouch 102 and a lower stomach pouch 104. The gastric restriction device 108 is adjustable by means of an implantable interface 110. A dynamic change imparted to the implantable interface 110 is transferred to the gastric restriction device 108 via a line 112.

In accordance with embodiments as disclosed herein, possible configurations for the implantable interface 110 include, but are not limited to, an injection port, an inductive coupling, a sonically activatable coupling, a magnetic coupling (consisting of permanent magnets and/or electro-magnets), and a compressible pressurization member (such as a diaphragm and valve system). Possible configurations for the line 112 include, but are not limited to, a fluid carrying tube, electrical conductors, a tension/compression cable-in-sheath system and a drive shaft-in-sheath system. All such variations of gastric restriction devices are compatible with the disclosed embodiments as described herein. Alternatively, the dynamic change can be imparted directly to the gastric restriction device 108, eliminating the need for the implantable interface 110 and the line 112.

While being viewed by X-ray fluoroscopy, the barium contrast suspension 116 is ingested by the patient, passes down the esophagus 106, through the lower esophageal sphincter 124 and into the upper stomach pouch 102. The upper pouch 102 empties into the lower stomach pouch 104, through a stomal opening 114 produced by the gastric restriction device 108. FIGS. 1 and 2, respectively, depict the stomach and contents before and after the barium suspension passes through the lumen of the stomal opening 114 that connects the upper and lower stomach pouches 102, 104 (also herein referred to as upper and lower, or proximal and distal, portions or regions of the stomach).

As used herein the terms “stomal opening” and “stomal lumen” are equivalent, and are intended to have their ordinary meaning, which includes, without limitation, the region between and connecting the two portions of a stomach formed when the stomach is engaged by a gastric restriction device 108.

By knowing the initial volume of the barium contrast solution 116 that was ingested, and by measuring the time for the upper stomach pouch 102 to empty, the flow rate through the stomal opening 114 can be calculated to be:

Mean Flow rate=(Volume of Barium Ingested÷Time to Empty)

For a 50 mL to 75 mL bolus of barium sulphate suspended in water at room temperature, an exemplary target mean flow rate is about 1 mL per second to about 20 mL per second, although flow rate is not necessarily limiting to the scope of the disclosure. It should also be noted that this is a mean flow rate. In some cases, it can be desirable to determine a “flow condition.” For example, a flow condition can be the presence or absence of flow through the stomal opening 114. The barium suspension can be warmed to about body temperature prior to ingestion by the patient in order to avoid significant viscosity changes due to warming after ingestion.

Accounting for the viscosity of the barium suspension 116, the effective diameter of the stomal opening can be calculated. As the level of barium suspension in the upper stomach pouch 102 decreases so too will the hydrostatic pressure that drives movement of the barium suspension through the stomal opening 114. The barium suspension can be warmed to body temperature prior to sipping, so that there is no significant viscosity variation due to warming after ingestion.

In the flow rate equation above, the mean flow rate is described. Note that as the upper pouch empties, the absolute flow rate decreases as the fluid level (and thus the driving pressure) decreases. For a given stomal opening size, it is expected that the mean flow rate will be at least in part related to the initial volume of the bolus ingested. Alternatively, residence time of the fluid in the upper stomach pouch can be a desirable measurement target, instead of mean flow rate or absolute flow rate. For example, where the restriction device provides an appropriate size opening, 30 mL of fluid would be expected to empty from the upper pouch in about four to six seconds.

Note that there is often variance in the effectiveness of a certain sized stomal opening from patient to patient. Whether a restriction device is providing the desired effect is typically a subjective determination based on patient feedback and in some cases observation by a caregiver. Different factors can affect the effectiveness of the restriction device. These include, among other things, a patient's own motivation to lose weight, a patient's tolerance to hunger and the quality of communication between the patient and their caregiver.

In addition, different patients may respond differently to a particular stomal opening size, and thus the most effective opening is likely to vary from patient to patient. For example, the most effective stomal opening internal diameter for weight loss can be 20 mm in one patient and 23 mm in another. Patient feedback as interpreted by a caregiver is one way in which stomal opening effectiveness is assessed. Patient feedback may include the amount of food that is eaten before the patient feels full, and the extent of vomiting that occurs if a patient consumes more food than the upper stomach pouch can reasonably hold. However, neither patient feedback nor caregiver observations are necessarily accurate measures of restriction device function. The present disclosure describes improvements to gastric restriction devices to provide the ability to determine a flow condition, to measure a flow rate, or to monitor the status of the restriction device with respect to such factors as gastric wall erosion or slippage of the device over time.

Traditionally, LAP-BAND® adjustments are performed or supervised by a bariatric surgeon. However, it is expected that by combining a noninvasive gastric restriction device adjustment means with the reliable method of flow detection provided by the present disclosure, a non-physician may at least perform flow testing and perhaps even the adjustment procedure. Moreover, the system described in the disclosed embodiment can alert the patient to early signs of restriction device slippage or gastric erosion so that timely follow-up by their physician can be sought at the earliest possible juncture.

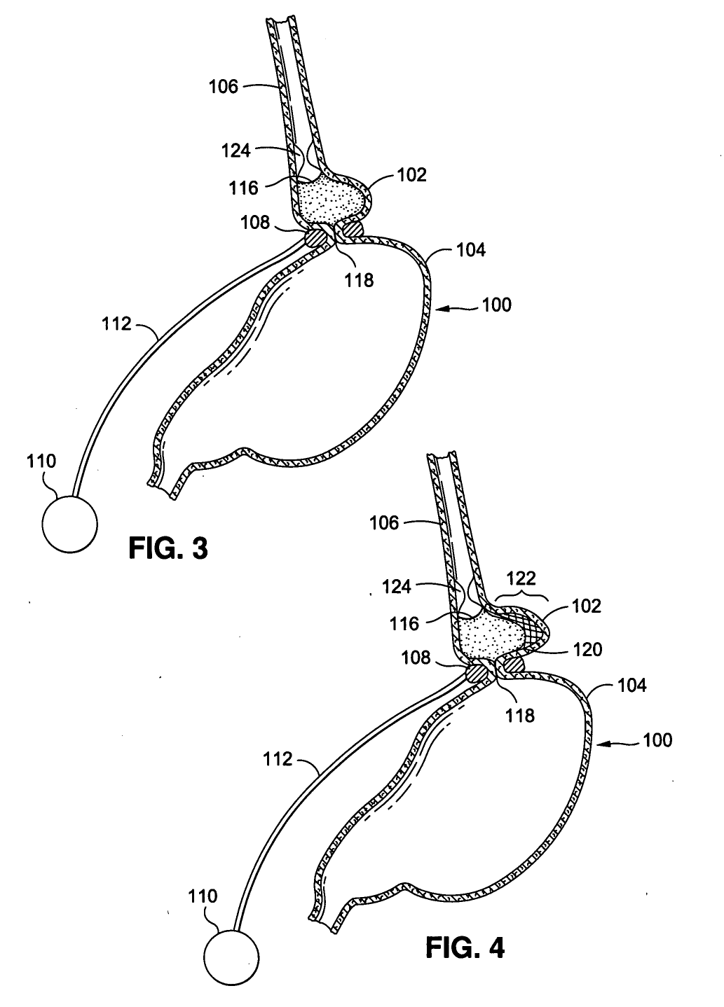

FIG. 3 illustrates one method of measuring the volume of the upper pouch 102, in order to determine whether any slippage of the device 108 or upper stomach pouch 102 growth has occurred. The gastric restriction device 108 is adjusted via the implantable interface 110 and the line 112 so that an occluded stoma 118 is created, and the patient's flow is effectively blocked. The patient now sips barium suspension in small gradations, for example, by drinking quantities of 10 mL until the upper stomach pouch 102 is seen to be full on X-ray (e.g., when the upper level of the barium contrast solution 116 is close to the lower esophageal sphincter 124). By knowing the total volume required to fill the upper stomach pouch 102, the general condition of the upper stomach pouch 102 can be deduced.

FIG. 3 illustrates an upper stomach pouch 102 that is at a desired volume. FIG. 4 illustrates an upper stomach pouch 102 that has grown undesirably due to slippage of the gastric restriction device 108 relative to the stomach 100. The area of slippage 120 translates into an enlarged portion 122 of the upper stomach pouch 102. The volume of the pouch obtained from the barium study can be correlated with the size of the radio-opaque area as observed by fluoroscopy.

Using these methods, the stability of the gastric restriction device 108 and its placement on the stomach 100 can be monitored from one adjustment procedure to the other. By combining this information with the comments from the patient, a desirable setting for the gastric restriction device can be determined. For example, the gastric restriction device may need to be tightened (to create a smaller stomal opening), loosened (to create a larger stomal opening), or the gastric restriction device may need to be repositioned or removed.

All of the methods described so far require the use of radiographic procedures such as fluoroscopy in order either to measure the volume of the upper stomach pouch or to monitor flow rate or residence of material in the upper stomach pouch. In addition, these methods are further limited in that they are only useful to follow materials that are detectable by radiography. Also, contrast suspensions, having significantly higher viscosities than water, do not demonstrate a quantifiable flow where the stomal opening has a very small aperture, and so it may not be possible to accurately adjust the restriction device to produce a very tight stomal opening, should that be desired.

Thus, some embodiments of the present disclosure provide apparatus and methods to monitor and adjust a gastric restriction device that avoid the use of X-ray fluoroscopy. These methods provide the further advantage in that they are noninvasive, involving the use of internally located monitoring means provided as part of the restriction device or placed during the surgical procedure to place the device in the patient, and simple enough for a patient or caretaker to perform the testing procedure. This simplifies and reduces the cost of testing, and enhances patient involvement in achieving their weight loss goals.

As used herein, the terms “internally mounted” or “internally located” are intended to have their ordinary meaning, which includes, without limitation, mounted or located within the body.

In some embodiments, the presence or absence of flow (i.e., a flow condition) or even a flow rate of a test substance through the stomal opening can be determined. In some embodiments, the method includes ingesting a known volume of a test substance detectable by a non-radiographic method, using a sensor means to detect the presence of the fluid at, or near, the stomal opening, producing an output from the sensor, and using the output signal from the sensor to monitor passage of the test substance through the stomal opening. Further, it is possible to determine the time it takes for known volume of the test substance to move through the stomal opening, and then if desired, calculate a flow rate of the test substance through the stomal opening.

As used herein, the term “sensor” is intended to include, without limitation, mechanical and/or electrical sensing devices, as well as the combination of sensing devices plus ancillary devices, for example, signal processors and controllers.

Thus, embodiments of the present disclosure describe alternative apparatus and methods to monitor and adjust the effectiveness of a gastric restriction device that avoid the use of X-ray fluoroscopy, and which can be adapted for use with either invasive or noninvasive means of adjusting a restriction device.

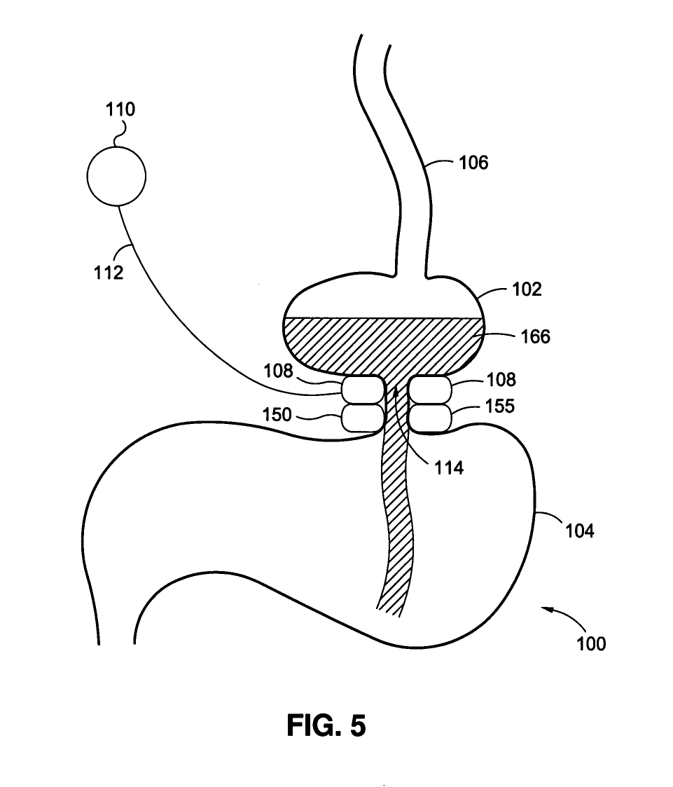

In the embodiment illustrated in FIG. 5, an internally mounted sensor 150 detects acoustic energy. The acoustic energy can be sound within the audible spectrum, ultrasound, or Doppler shift echoes produced from ultrasound. The sensor 150 is used to monitor flow of an ingested substance, for example a sound-producing fluid 166, through the stomal opening of a gastric restriction device 108. The sensor 150 can be an internally placed microphone, pickup, or any other suitable means of detecting sound, without limiting the scope of the disclosure. The sensor is capable of detecting the sound-producing fluid as it moves from the upper stomach pouch 102 to the lower stomach pouch 104, through the stomal opening 114. The sensor 150 may be included as an integral component of the gastric restriction device 108, or alternatively, may be separate from the restriction device 108. The precise location of the sensor 150 is not critical to the operation of the system, as long as the location is such that the sound-producing fluid is detectable by the sensor 150.

Signal data from the sensor is relayed outside the patient via a telemetry unit 155. Interpretation of the output signal from the sensor 150 provides information about flow conditions through the stomal opening 114. In some embodiments, it is desirable to determine a flow-versus-no-flow condition through the stomal opening. In other instances, it may be desirable to determine flow duration, residence time of the fluid in the upper stomach pouch 102, or even flow rate. In either case, information obtained regarding flow through the stomal opening 114 can be used to adjust the restriction device 108 via an implantable interface 110 to provide a desired flow condition or flow rate.

A line 112 connects the interface 110 to the device 108. The line 112 may be a cable to transmit an electrical signal to a drive mechanism provided as part of the device 108, or may be a drive shaft-in-sheath operative to vary the aperture produced by the device via a transmission in the device, which in turn will vary the size of the stomal opening 114. The line 112 can also be a pressurized line to vary the inflation of a bellows or other such aperture regulator included as part of the device 108. The choice of interface, line or means for varying the size of the restriction device aperture is not limiting to the scope of the disclosure.

In the embodiment illustrated in FIG. 5, the sound producing fluid 166 can be water, and the sound detected is the sound that the water makes as it flows through the stomal opening 114. The stomal opening produced by the gastric restriction device is analogous to a sphincter, and as water squirts through the opening urged by gastric peristalsis, detectable sounds will be produced. Alternatively, the sound-producing fluid may comprise an effervescent solution including effervescent granules taken with water, for example a mixture of sodium bicarbonate and tartaric acid in water. Other effervescent solutions are also compatible with the present disclosed embodiments, and so the specific composition of the solution is not limiting. For example, the solution may comprise gas-producing substances such as carbon-dioxide embedded candies as described in U.S. Pat. Nos. 3,012,893; 3,985,709; 3,985,910; 4,001,457; 4,289,794, all of which are incorporated herein by reference.

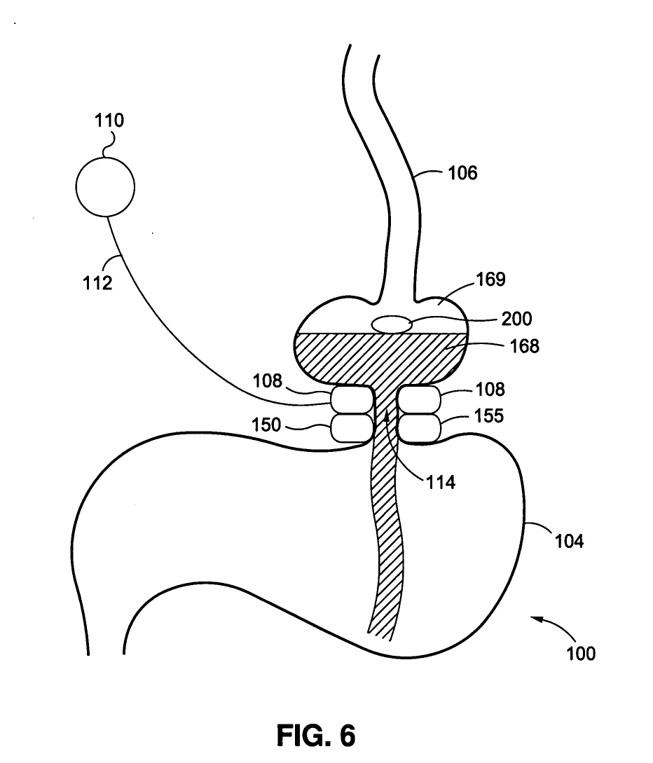

In some embodiments, illustrated in FIG. 6, the “sound-producing fluid” can be an ingested substance 168, further comprising a sound-producing capsule 200, such as that disclosed in U.S. Pat. No. 7,160,258, the entirety of which is incorporated herein by reference. The capsule 200 may be biodegradable, or alternatively, it can be biocompatible such that is passes safely through the body. The capsule 200 may be free in solution such that it passes through the digestive tract and is eventually expelled, or secured by a line or tether to provide for removal from the patient immediately at the end of a test session. The capsule 200 can be chosen such that its density is less than that of the ingested substance 168, so that the capsule floats at the surface of the ingested substance. A floating capsule effectively marks the interface between the ingested substance and the adjacent airspace 169. Conveniently, the ingested substance may comprise a fluid such as water or any other suitable fluid.

The sound produced by the capsule 200 may be in the audible range or may be ultrasonic or subsonic, depending on the nature of the sensor employed. In addition, the acoustic signature of the capsule may be selected in order to more easily distinguish the sound of the capsule from normal body sounds, such as those occurring in the heart and circulatory system as a result of breathing or due to normal peristaltic action or trapped gases in the gastrointestinal tract. Likewise, if desired, during the course of the test, the sound of normal body noises may be subtracted from the output signal using an active noise cancellation technology that discriminates between the acoustic output of the capsule and other noises.

Similar improvement in detection can also be provided by a band pass filter to limit the frequencies detected to those most characteristic of the particular sound-producing fluid being employed. The sound processing capabilities may be provided as part of the telemetry unit 155, or may optionally be provided as part of an external receiver. Using these methods either alone or in combination, the signal to noise ratio is effectively increased, and the top of the fluid level is sensed while it is in the upper pouch until it passes through the stoma opening. Methods of acoustic filtering or noise cancellation, while useful in conjunction with some embodiments, are not essential to the operation of the disclosed embodiments as described herein, nor are they to be considered limiting to the disclosure. Alternatively, the capsule 200 can be configured to transmit radiofrequency transmissions, which can be sensed externally in an analogous manner.

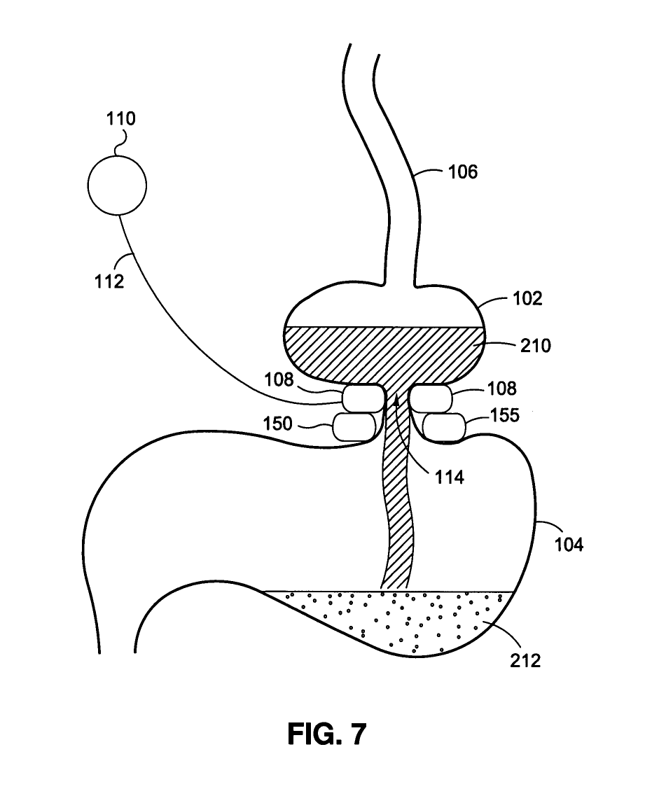

In some embodiments, like that shown in FIG. 7, where an effervescent solution 210 is being monitored, an additional variation in the procedure may be added to improve the accuracy of determining when the solution has passed from the upper stomach pouch 102, through the stomal opening 114, and into the lower stomach pouch 104. In this case, a pH-buffered solution 212 is first ingested and allowed to fill at least a portion of the lower stomach pouch prior to the drinking of the test substance, which comprises an effervescent solution 210. The pH of the buffered solution 212 is selected such that it neutralizes the effervescent solution when the two are mixed. As the effervescent solution passes through the stomal opening 114 into the lower stomach pouch 104, it will mix with the pH-buffered solution 212. The mixing of the two solutions in the lower stomach pouch will result in rapidly reduced effervescence, resulting in a similarly rapid decrease in sound levels, in turn leading to more accurate determination of when the contents of the upper stomach pouch have substantially emptied into the lower stomach pouch, due to elimination of significant residual sound.

The sensor 150 produces an output directly related to the intensity of the sound detected. Output from the sensor 150 can be relayed externally by a telemetry unit 155.

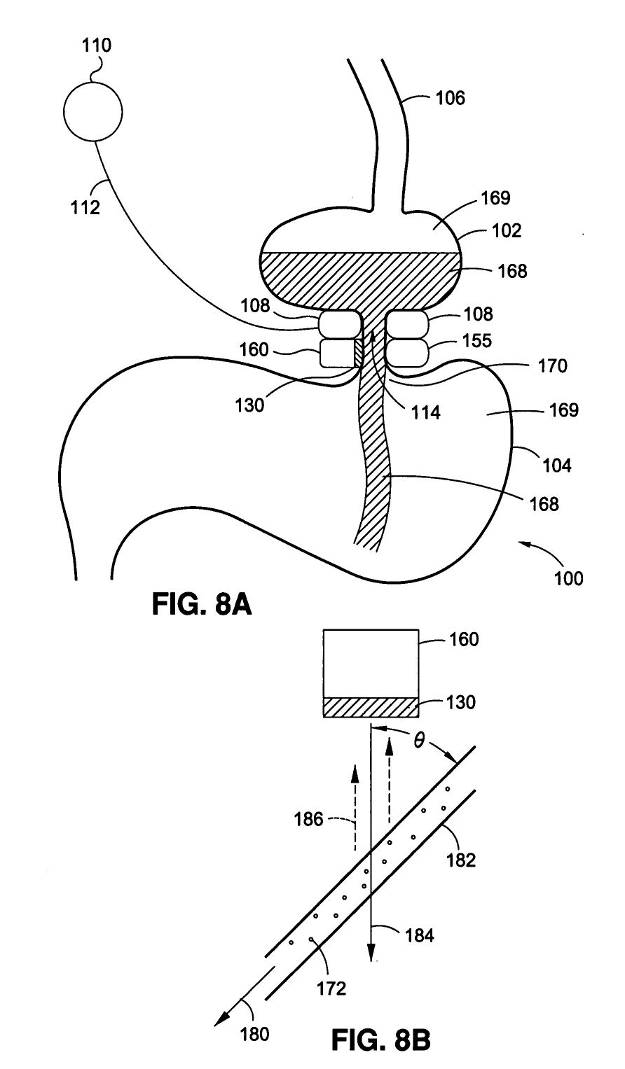

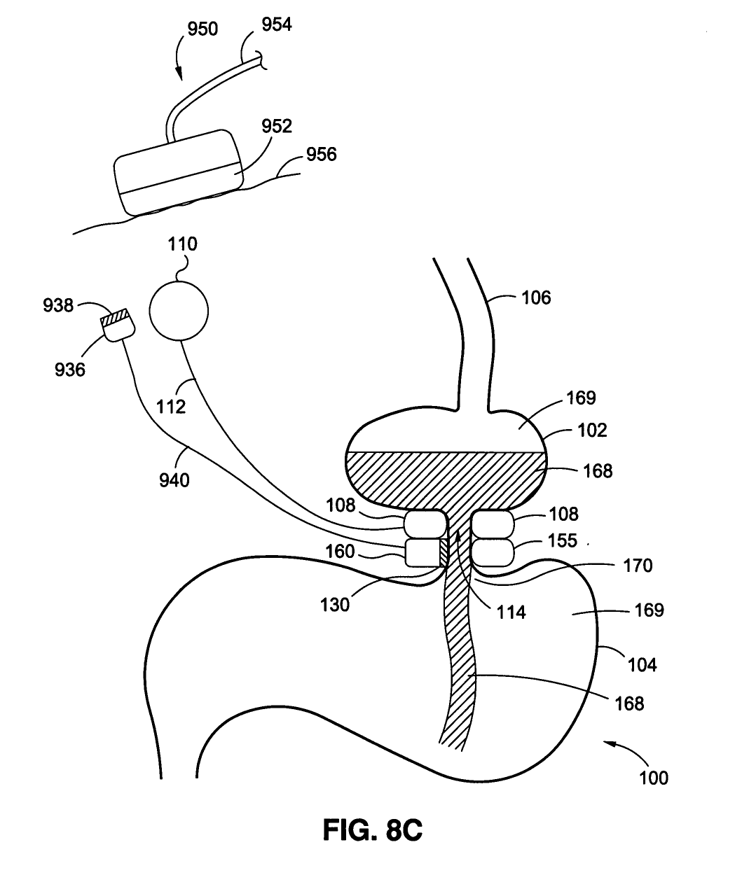

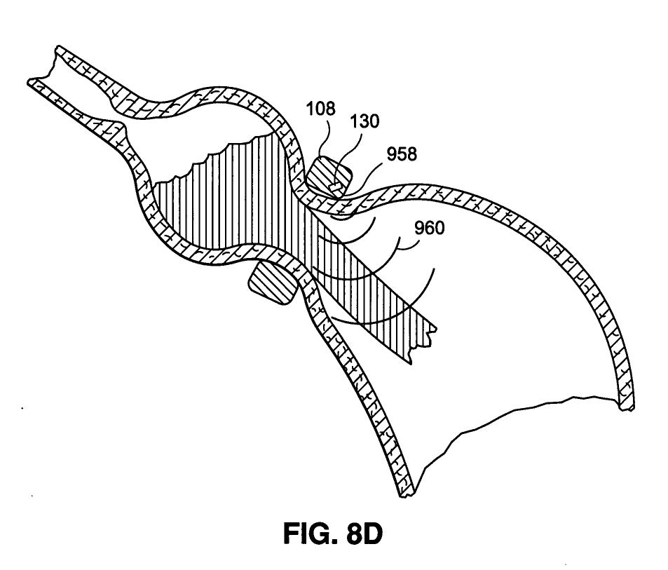

In addition to simple detection of sounds produced by an ingested substance, methods of measuring flow, flow rate (for example, volumetric flow rate and/or mass flow rate), velocity, or residence time, based on Doppler ultrasound, are also contemplated in the present disclosure. For example, as illustrated in FIG. 8A, an internally-mounted Doppler ultrasound probe 160 with transducer 130 uses ultrasound to detect movement of a test substance 168 from the upper stomach pouch 102 to the lower stomach pouch 104 through the stomal opening 114 produced by the gastric restriction device 108.

Ultrasound transducers are well-known in the art. For example, a transducer like that available from Measurement Specialties, Inc., made from Polyvinylidene Fluoride (PVDF), and described in U.S. Pat. No. 6,504,289, herein incorporated by reference, could be adhered to the inner surface of the restriction device 108 or placed immediately next to the device, as illustrated. Alternatively, the ultrasound transducer could be located separate from the gastric restriction device. The precise location of the ultrasound transducer is not critical to operation, as long as the location is such that the ultrasound transducer can effectively permit the detection of the test substance as it moves from the upper stomach pouch to the lower stomach pouch through the stomal opening.

In some embodiments the transducer 130 is configured to vibrate at a frequency in a range of from about 1 MHz to about 30 MHz. In some embodiments the transducer is configured to vibrate in a range from about 5 MHz to about 15 MHz. An angle θ is defined as the angle of incidence between the pulses and the direction of fluid flow 180, for example in a tube 182, as illustrated in FIG. 8B. Scattering agents 172 enhance the production of return echoes 186. If the transducer frequency is defined as fₜ then the Doppler shift frequency (f_{d}) is:

$${f}_{d} = \frac{2 {f}_{t} V cos θ}{c}$$

where c is the speed of sound in tissue and V is the measured velocity of the fluid or object in motion. Solving for velocity:

$$V = \frac{{f}_{d} c}{2 {f}_{t} cos θ}$$

Depending on the acoustic impedance of the material into which the output pulses are directed, the ultrasound output 184 may generate return echoes 186, as in FIG. 8B. Return echoes are most efficiently created when there is a difference in the acoustic impedance (i.e., an impedance mismatch) between two regions or materials. For example, a stomach completely filled with pure water is not very effective to produce Doppler shift echoes from ultrasound, as the acoustic impedance of water is very similar to that of skin, fat, muscle, and other body tissues. In contrast there is a significant difference in acoustic impedance between fluid contained in the stomach and an adjacent air or gas region, as would occur when the stomach is less than completely full. In addition, where a fluid further comprises objects or particles that scatter the ultrasound energy, an enhancement of return echoes will be observed. For example, crystals of barium sulphate suspended in water are effective to scatter ultrasound.

Medical Doppler systems take advantage of the Doppler effect, in which a Doppler frequency shift (the difference between the original ultrasound pulse frequency and the return frequency) provides information about relative motion. The typical velocities of fluids being probed in medical applications create Doppler shifts with frequencies that lie within the audible spectrum (i.e., 20 Hz-20 kHz). This sound can be calibrated to provide a flow velocity, as is done in cardiac ultrasound applications. In the case of a gastric restriction device, it is not always possible to directly derive flow rate from flow velocity. This occurs primarily because the aperture of the gastric restriction device is not necessarily predictive of the actual size of the stomal opening that it produces in vivo. This occurs due to variability in stomach wall thickness, as well as in the precise location of the restriction device from patient to patient. Testing has shown that the fluid motion through the stomal opening can be detected using a Doppler ultrasound instrument.

Thus, some embodiments take advantage of the difference in acoustic impedance at the interface 170 between the test substance 168 and the adjacent airspace 169 as a means of “marking” and monitoring the progress of the interface 170 between the two as the substance 168 in the upper stomach pouch 102 moves to the lower stomach pouch 104. Thus, while a simple fluid such as water is relatively poor in terms of providing a media for distinguishable return echoes, echoes are produced as the ultrasound signal encounters the interface between the fluid and the adjacent airspace, and these can be received by the transducer and outputted as a useable signal. The signal from the ultrasound probe 160 can then be relayed via a telemetry unit 155 to an external receiver for display, recording, and further processing of the data obtained.

With respect to adjusting a gastric restriction device, there are at least two forms of output that will generally be useful. First, detecting a flow versus no flow condition can be effective to allow adjustment of the device. For example, in some embodiments it may be desirable to adjust the restriction device so that it is in a substantially closed position, thus providing little or no opening between the upper and lower stomach pouches, and then open the device just until a flow is detected. This would provide a fairly aggressive adjustment of the device, but would result in more effective weight control as the amount of food a person could consume comfortably would be quite small.

In contrast, the desired output can be an average flow rate, calculable from the flow duration (i.e., the time from which a volume of test substance begins to flow through the stomal opening to when it has completed flowing through the stomal opening). As with the sound detecting embodiments, an automated timing mechanism can start and stop a timer based on pre-determined threshold values in order to determine a time interval based on detection of the test substance as it flows from the upper stomach pouch to the lower stomach pouch. Knowing this time interval and the volume of the test substance ingested, the following calculation will yield an average flow rate:

Flow rate(mL per second)=Volume(mL)/Time(sec)

Alternatively, calculations can be done manually by manual timing and manual calculation or by using a computer processor 504 as described below.

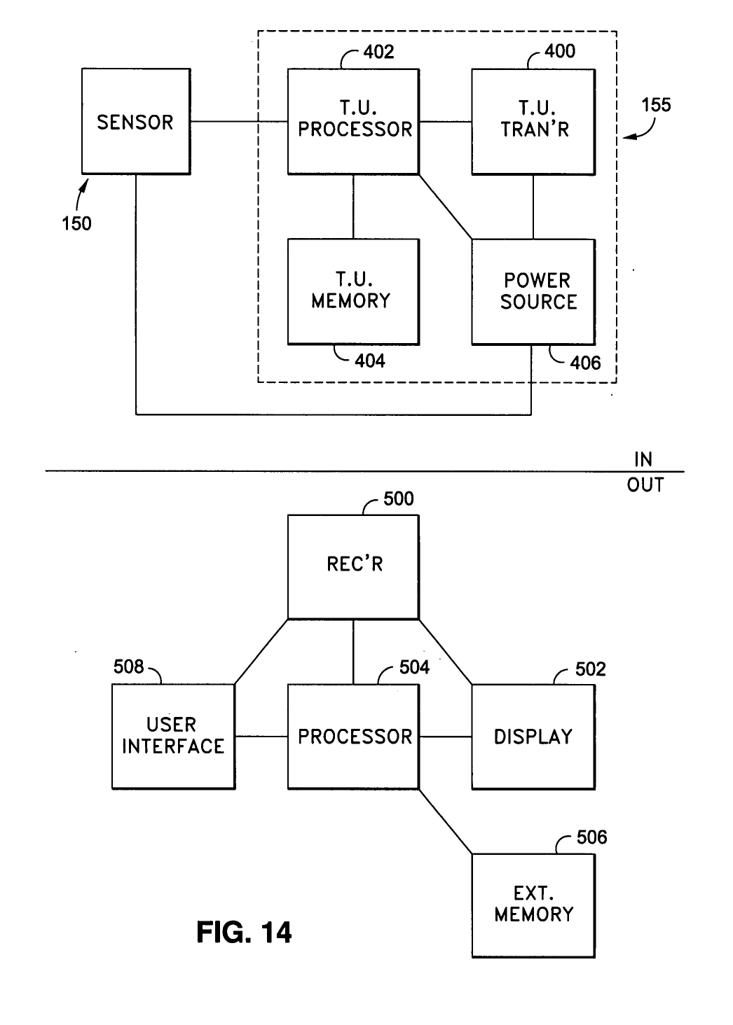

A computer processor 504 and display 502, schematically shown in FIG. 14, also provide additional functionality, such as being able to program in the volume and viscosity of the test substance. Even more elaborate data processing may include a programmable correction function to account for situations where the test substance is at a temperature other than body temperature in order to provide a corrected flow rate. The computer processor 504 can also be linked to a user interface 508, and an external memory 506 adapted to store either programming instruction or to receive data from one or more test sessions.

Referring to FIG. 8A, in situations where the flow rate measurement is conducted using water as the test substance 168, detection will generally be achieved where the Doppler transducer 130 is directed towards the interface 170 between the water and the stomach airspace 169. The disclosed embodiments thus also provide for a transducer that is relatively easy to orient at the time the gastric restriction device is surgically implanted. For example, where the transducer is integral to the restriction device, there may be provided a means of rotating the transducer such that it points in a desired direction. In some embodiments, an integral transducer may be located in the gastric restriction device such that upon placement of the restriction device the transducer will be in an effective orientation, as shown in FIG. 8D. Further, in some embodiments a plurality of transducers, arranged as a generally circumferential array near the stomal opening can provide an even more effective ultrasound-based sensing system.

FIG. 8C illustrates an embodiment of a passive ultrasonic system for Doppler flow measurement. Coupled to the Doppler transducer 130 via a conductor 940 is a Doppler probe 936 having a second Doppler transducer 938, configured to be implanted in the patient, for example subcutaneously or intra-abdominally. The Doppler probe 936 can be secured to the fascia at an internal or external portion of the abdominal wall, for example, with suture, staples, spiral tacks, or analogous fasteners. In this configuration, the implanted Doppler instrument requires no active electronics to power it. Power is applied from the outside of patient via an external Doppler probe 950 placed on the patient's skin 956. A coupling gel between the one or more transducer elements 952 and the skin 956 is used for impedance matching.

A signal is transferred through a conductor 954 to the transducer elements 952 resulting in oscillation of the transducer elements 952. The ultrasound pulses which are created are propagated through the skin and fat to the second Doppler transducer 938 of the Doppler probe 936, resulting in oscillation of the second Doppler transducer 938. This oscillation produces a signal which is then transferred through the conductor 940 to the Doppler transducer 130 of the restriction device 108. This results in oscillation of the Doppler transducer 130, producing a pulse in the area of the stoma.

When echoes are received, the process happens in reverse. The echoes result in oscillation of the Doppler transducer 130, producing a signal that travels through the conductor 940 to the second Doppler transducer 938 of the Doppler probe 936. The oscillation that is created in the second Doppler transducer 938 results in a pulse that is propagated through the fat and skin 956 to the transducer elements 952 of the external Doppler probe 950.

This embodiment provides several advantages, including obviating the need for implanted active electronics. As no control system and no power, such as a battery, are needed in the implanted portion of the system, the implant can be manufactured at lower cost, and in addition is more durable and reliable.

FIG. 8D illustrates an embodiment of a restriction device 108 having an embedded Doppler transducer 130. The material 958 that covers the Doppler transducer 130 is a matching layer, comprising a material having good impedance matching, such that the device effectively conducts ultrasound. In one embodiment, an angled arrangement of the Doppler transducer 130 in relation to the restriction device 108 allows the pulses 960 to travel in a minimal angle in relation to the flow of the test substance 168, as shown in FIG. 8B.

Variations in flow rate, or flow condition, that significantly depart from otherwise normal variability provide an early indication that the restriction device is not functioning properly, has slipped from its implantation site, or needs to be adjusted to maintain a desired flow rate through the restriction.

Storing data from multiple test sessions can be of use to a physician who is monitoring a patient's status over a period of time. Furthermore, other problems related to the use of gastric restriction devices, such as gastric erosion, might be detected earlier, allowing the physician to intervene at a relatively early time to avoid more serious complications.

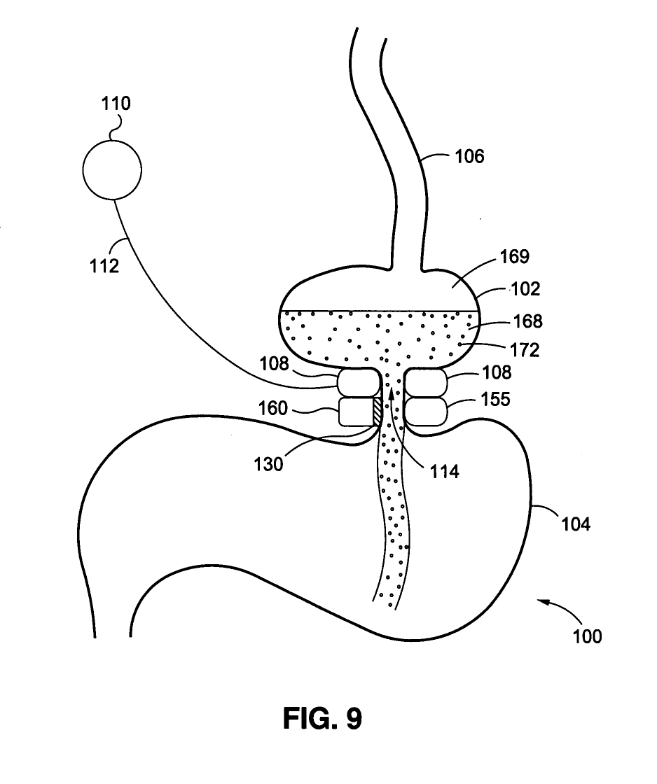

In some embodiments, one of which is illustrated in FIG. 9, the test substance 168, for example, a fluid, can optionally include at least one scattering agent 172. Scattering agents are effective to scatter ultrasound waves and increase the production of Doppler shifted return echoes. Scattering agents suitable for use with ultrasound systems are well known in the art and may include, without limitation, such items as flax seed, micro-bubbles, micro-spheres, or Kaolin clay. The use of these scattering agents within the test fluid provides an acoustic impedance difference in the test substance itself as compared to surrounding tissue, instead of only at the fluid/gas interface in the stomach.

Barium sulfate is generally insoluble in water, existing as a suspension of microscopic particles, which also will effectively enhance echo generation when probed by ultrasound. Thus, barium sulphate particles present in a barium contrast solution are also effective to scatter sound waves and enhance the signal perceived by the Doppler device. The use of scattering agents in the ingested test substance improves direct detection of fluid movement through the stomal opening where there may not be a sufficient fluid/gas interface, or where there is an insufficient impedance mismatch. Improving fluid detectability also makes placement of the transducer less critical. This further simplifies either placement of the sensor system where the transducer is separate from the restriction device or the design of the transducer-restriction combination where a transducer integral to the restriction device is used.

It should be noted that while it is an object of the disclosure to be able to accurately adjust a gastric restriction device without resorting to the use of radiographic techniques, the present method and apparatus could be advantageously used in conjunction with other methods of evaluating flow use barium swallow and X-ray fluoroscopy. Use of these techniques would provide for visualization of flow, while listening for characteristic sound signatures from the Doppler. Such a combination can be useful when training new users of the system in recognizing the correlation between sound output of the Doppler and movement of material through the stomal opening, or when calibrating or programming the sensor apparatus. Providing a visual correlation to the sounds detected would improve the acquisition of skills needed to perform a flow test with acceptable accuracy.

Some embodiments provide an accurate measure of flow rate through the stomal opening produced by a gastric restriction device. However, depending on the nature of the material being consumed (e.g., fluid or food) flow rate may vary. For water, a desired flow rate might range from about 1 mL to about 20 mL per second, or in the range of from about 5 mL to about 15 mL per second. In contrast, a more viscous solution such as a BaSo₄ suspension in water will have a slower flow rate, proportional to the amount of barium in the suspension. In the instance where the restriction device has been adjusted to provide a very small opening, very little flow of a viscous material may result, a condition that will be readily detected by embodiments of the present disclosure.

BaSo₄ suspensions are commercially available, for example E-Z-PAQUE®, and have viscosities ranging from about 400 cP to about 750 cP over the typical flow rates encountered in clinical applications. Solutions with even higher viscosity will be expected to move even more slowly through the opening. For example, it is known that solid food may be blocked by a stomal opening where liquids like water will readily pass. Therefore, in some embodiments of the disclosure there is provided a means of measuring flow rate or flow condition with solutions having varying viscosity in order to better model the behavior of the various foods or beverages that the patient might normally consume, and thus derive a desired flow rate.

This may be accomplished through the use of test substances of varying viscosity in order to mimic the flow rate of a variety of ingested materials. For example water at 20° C. has a viscosity of 1 cP. Solutions with varying amounts of sucrose present can have viscosities ranging from about 3 cP to about 3,000 cP. Vegetable juices can have viscosity values ranging from less than about 10 cP to greater than about 3,000 cP. Solid foods have even higher viscosity values, as high as about 1×10⁵ cP or even greater. Thus a low viscosity test substance might be one with a viscosity of less than about 10 cP, a medium viscosity test substance might be in the range from about 10 cP to about 10,000 cP and a high viscosity substance might have a viscosity from about 10,000 cP and higher.

Thus, in terms of usefulness of the data obtained in testing flow rates, or even to a flow condition, it will be desirable within a test session to evaluate flow or flow rate for substances of differing viscosity, not only to check for flow through the stomal opening, but to ensure that the opening can accommodate desired rates of flow over a range of substance viscosities typical of fluids and foods ingested by most people. For greater certainty regarding the function of the restriction device, low, medium and high viscosity test substances or fluids may be tested in turn as part of a single testing session, and in this way the most beneficial adjustment of the gastric restriction device may be made based on a desired flow rate. As the test is relatively easy, noninvasive and of short duration, testing multiple fluids would not be particularly burdensome to the patient, and would potentially provide the physician or other caretaker with the best possible information as regards the functioning of the gastric restriction device in order to adjust the device to provide a desired flow rate.

Water is useful as a test fluid, especially when testing highly constricted stomal openings, as water has a relatively low viscosity and will flow relatively unimpeded through whatever stoma is provided. Viscosity is also affected by the temperature of the material, such that as temperature increases viscosity typically decreases. For example, water has a viscosity of 1 cP at 20° C., which decreases to about 0.69 cP at 37° C. Thus, it is advantageous to provide a means of equilibrating the test fluid to a pre-determined temperature prior to ingesting in order to reduce test to test variability. For example, the test fluid can be equilibrated to a temperature about the same as the patient's body temperature (typically about 37° C.) in order to minimize changes in fluid viscosity that would otherwise occur as the fluid warms in the body after ingestion.

Embodiments of the present disclosure provide a means for determining a flow condition, which includes, without limitation, determining whether or not material moves past, or through, the stomal opening. Flow condition is a qualitative measure. However, in all of the modalities described above, embodiments can be further adapted to provide information about flow rate by including timing means that is activated when the relevant sound is sensed above a pre-determined threshold level. Likewise, the timer may be stopped when the relevant sound drops below the threshold intensity. By combining time measurements and the volume of material ingested, an accurate calculation of flow rate past the restriction device can be determined. The timing mechanism may further be under the control of a processor such as that described below. In some embodiments, the output from the Doppler ultrasound may also be saved as a computer file using a sound analysis software program, and the data analyzed at some point in the future.

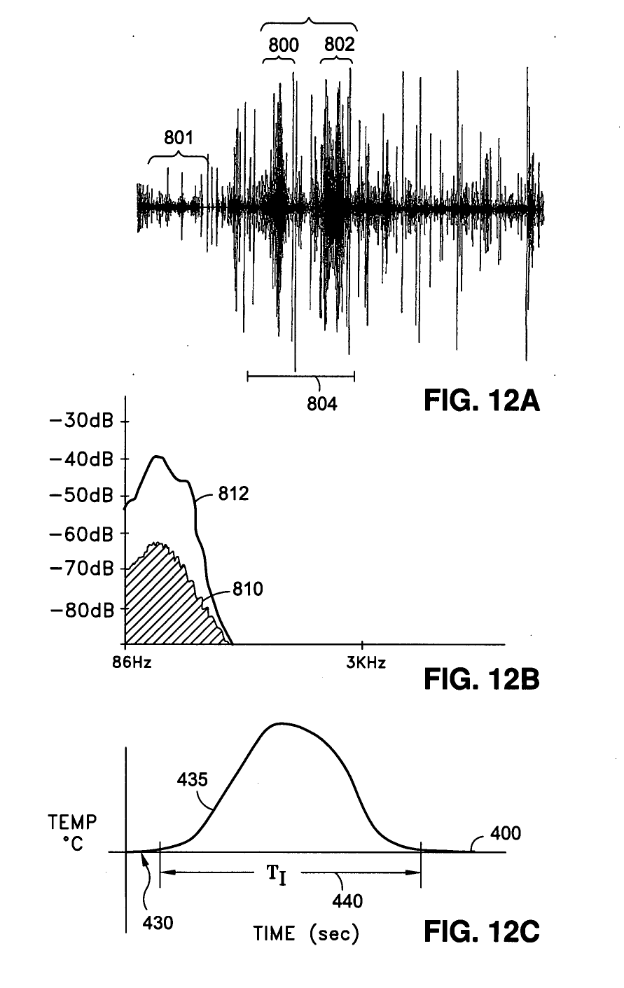

An example of a sonogram from a Doppler ultrasound experiment is shown in FIG. 12A. While this data was collected using an externally located ultrasound transducer, it nonetheless illustrates the basic principles of the disclosed embodiments, which are generally applicable to Doppler ultrasound. As can been seen from these data, movement of fluid through the stomal opening occurs in a pulsatile fashion, influenced by peristaltic contractions. As shown, two periods of increased sound intensity 800, 802 were observed. By comparison, background sounds 801 not related to movement of fluid through the stomal opening are detected but at appreciably lower levels. Barium fluoroscopy performed concomitantly confirmed that movement of fluid from the upper stomach pouch to the lower stomach pouch coincided with the periods of increased sound intensity 800, 802.

From this, a time interval 804 can be calculated corresponding to the time it takes a volume of material in the upper stomach pouch to move through the stomal opening into the lower stomach pouch. Dividing the total volume ingested by the time period provides an average flow rate. Spectral analysis of baseline 810 and fluid movement-based 812 Doppler echo returns, as in FIG. 12B, shows that during movement of fluid through the stomal opening, not only does intensity of Doppler return echoes increase, but that return signals have distinguishable spectral characteristics (notice the shoulder on the right portion of curve 812, as compared to curve 810).

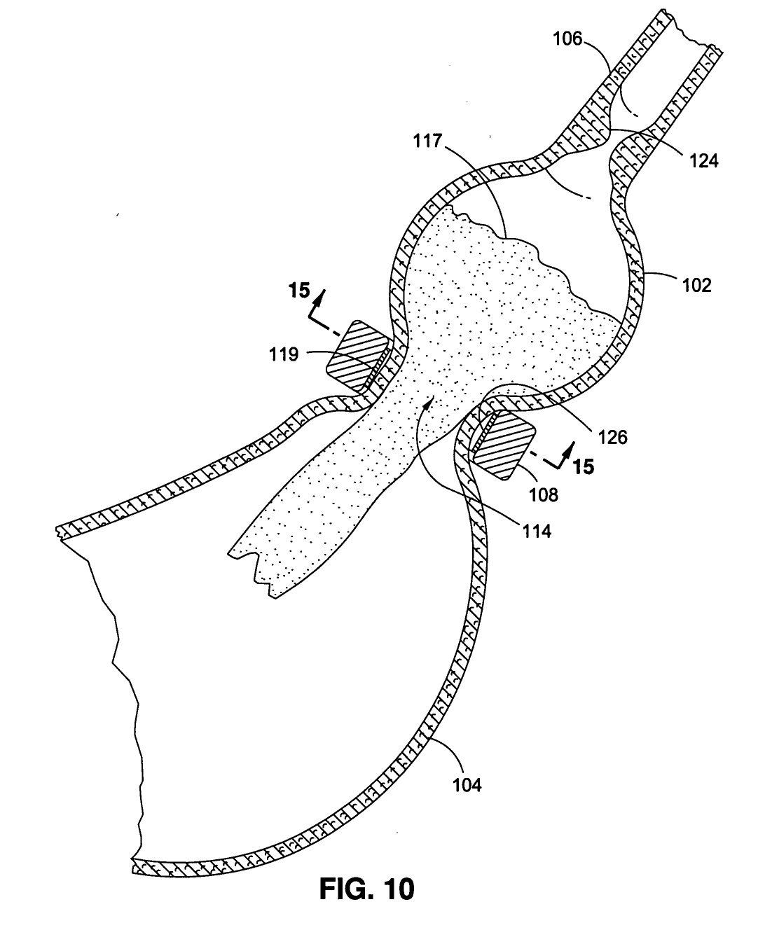

As an alternative to monitoring of sound-producing fluids, an internal sensor capable of detecting a physical or chemical property of an ingested substance can be employed. For example, in some embodiments the capacitance of a fluid that is swallowed by the patient is measured by a capacitance sensor, integral to the restriction device, as shown in FIG. 10. The ingested substance 168 can, in some embodiments, be a fluid, and in particular plain water, depending on the choice of sensor and the electronic circuitry provided to process the sensor output.

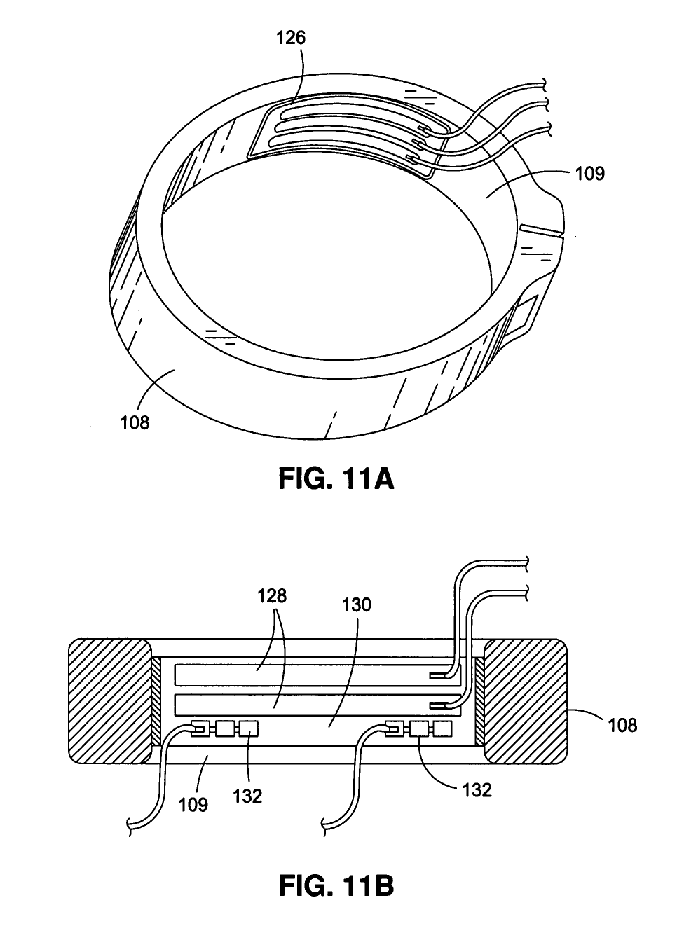

FIGS. 11A and 11B illustrate some embodiments of a capacitance sensor 126 integral to the gastric restriction device 108. Capacitance sensor electrodes 128, for example, electrodes fashioned from palladium alloys or other biocompatible metals, are secured into a flexible polymer substrate 130 (for example, polyimide) and then anchored to an inner surface 109 of the restriction device 108. In some embodiments, electrodes 128 cover a limited portion of the circumference of the inner surface of the gastric restriction device 108. In some embodiments this includes the portion of this circumference that is relatively non-dynamic, or that does not significantly constrict or contract, in order to maintain relatively consistent contact, for example, the latch which is used to close the band around the stomach.

In some embodiments, the electrodes 128 are positioned close to the gastric wall, but are generally electrically isolated from the gastric wall and from each other. The precise design of the capacitance sensor is not limiting to successful operation of the system, and those skilled in the art will readily recognize a number of designs suitable for such sensors. For example, FIG. 11A shows one such arrangement where the sensor electrodes 128 extending less than 90° around the interior of the gastric restriction device 108. In some embodiments, the sensor electrodes are configured to extend axially.

In some embodiments of a method using such a sensor system, the patient begins by drinking a known volume of a test substance, for example, a high capacitance fluid 117, as shown in FIG. 10. Capacitance is proportional to the dielectric constant of the test substance. Distilled water has a dielectric constant of about 74 at 37° C. (i.e., body temperature), whereas air has a dielectric constant of slightly greater than 1 (by definition a vacuum has a dielectric constant=1). A solution of barium titanate (BaTiO₃) can be used as the test substance instead of water. Pure barium titanate has a dielectric constant ranging from 90-1250 depending on temperature. Certain solutions of barium titanate in water have been demonstrated to be non-toxic in mice and rats, as described in U.S. Pat. No. 4,020,152 (issued to Heitz; the contents of which are herein incorporated by reference in their entirety). In some embodiments, a solution of titanium dioxide (TiO₂) can be used. Titanium dioxide has a dielectric constant ranging from 80-110.

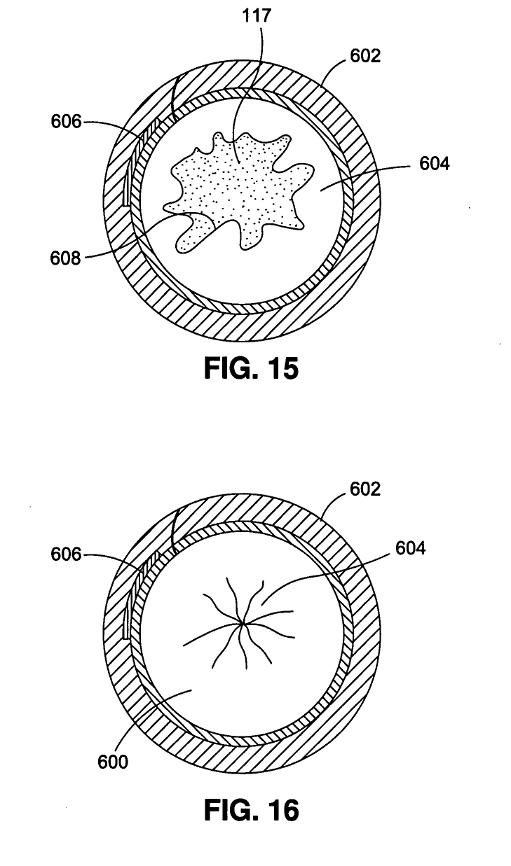

In some embodiments, the sensor 126 is configured to detect the test substance as soon as it begins to pass through the stomal opening. The sensor 126 detects the change in local capacitance, for example, an increase in local capacitance resulting from the presence of the high capacitance fluid 117 within the stomal opening. Capacitance will also vary depending upon the flow rate of the test substance, as will be understood by comparing the cross-sectional appearances of FIG. 15 and FIG. 16. FIG. 15 is a section taken from FIG. 10 while the test substance is passing through the stomal opening, while FIG. 16 is a section taken from the same location when there is no flow, for example, prior to ingestion of a test substance.

Often, when a gastric restriction device is adjusted to a desired condition, the stomach wall assumes the configuration shown in FIG. 16. With nothing ingested and no significant peristalsis taking place, the stomach wall 600 is compressed enough by the gastric restriction device 602 so that the stomach wall 600 substantially closes the stoma 604, similar to the shape of a sphincter. Electrodes 606 sense one or more electrical properties related to capacitance.

In contrast, and as shown in FIG. 15, a high capacitance fluid 117 flows through the stomal opening 608. Flow will typically occur due to peristalsis that normally occurs following ingestion. At this point, the capacitance of the contents inside the gastric restriction device 602 is a combination of the capacitance of the compressed stomach wall 600 and the capacitance of the high capacitance fluid 117. If the dielectric constant of the high capacitance fluid 117 is higher than the dielectric constant of the stomach wall 600, the total capacitance increases proportionally with the amount of high capacitance fluid 117 that flows through the stomal opening 608.

Typical dielectric constants of body tissue are presented in Table 2. The sensor 126 produces an output signal that can be relayed via a telemetry unit 155 to receiver 500 located outside the patient's body (FIGS. 10 and 14). In the simplest state, the output signal from the sensor 126 signals a flow versus no-flow condition. As described above, in some embodiments, a method of adjusting a gastric restriction device comprises completely restricting flow, then opening the aperture just enough so that flow is detected. The presence of flow versus no flow can be indicated by a display including, without limitation, illumination of color coded LEDs, generation of an audible tone, or other like simple “on-off” type displays. In some embodiments, a capacitance drop can be measured, for example, when using a test substance having a low dielectric constant (e.g., air).

TABLE 2
Dielectric Constant of Body Tissues

| Body Tissue | Dielectric Constant |
|---|---|
| Fat | 16 |
| Striated or smooth muscle | 50-60 |
| Bone | 15-25 |
| Blood | 58 |

In some embodiments, the detection of flow can be used to activate a timer. Timer functionality could be included as part of the telemetry unit processor 402 or the external processor 504, as desired. Once the volume of the high capacitance 117 fluid had completely passed through the stomal opening, capacitance would return to normal or near normal. In this case, a no-flow condition can be indicated, or the timer can be stopped, yielding an elapsed time measurement. As with other embodiments, there would be provided a telemetry unit 155 to relay the data from the sensor to an outside receiver 500. Timing information can be used where it is desired to determine an average flow rate in addition to merely ascertaining the presence or absence of flow through the stoma.

As has been described, knowing the volume of material ingested, and the time taken to pass through the stomal opening, an average flow rate can then be calculated. Other configurations are also possible, and thus the precise configuration is not meant to be limiting. For example, some embodiments include a sensor that is internally mounted but not integral to the gastric restriction device. Positioning of the sensor is not critical to the operation of the system so long as the sensor is within adequate proximity to sense passage of the test substance through the stomal opening. In some embodiments, there may be provided an array of multiple sensors arranged circumferentially around the stomal opening in order to provide the most accurate sensing of flow. This could be especially useful when it is desired to adjust the stomal opening to a minimal aperture size that still permits some flow. An array of multiple sensors would be expected to be especially sensitive and able to detect very low flow rates.

Examples of circuitry that could be adapted for use in some embodiments as presently disclosed are provided in U.S. Pat. Nos. 4,099,118 (Franklin et al.); 4,464,622 (Franklin); and 6,023,159 (Heger); the contents of all of which are incorporated herein by reference. In each of the cited examples, the fundamental operating principle is that the dielectric constant of an object will directly affect the capacitance of a capacitor plate placed on or near the object. As the capacitor plate is moved from one location to another, changes in the dielectric constant of the material will be detected as variations in capacitance.

In the present disclosure, the same principle of operation has been adapted for use in detecting the capacitance of fluid flowing through the stomal opening of a gastric restriction device in order to be able to detect flow and measure flow rate, such that adjustments can be made to the restriction device. Here, the relative motion of a moving fluid past a stationary capacitance sensor will serve to provide information as to the presence or absence of a test substance at the stomal opening. As fluid moves through the stomal opening, it will cause a change in the local dielectric constant that can be detected by a capacitance plate sensor system analogous to those described above.

Simple detection of the high capacitance fluid in the stomal opening can readily distinguish qualitatively between flow and no flow conditions, and will be useful where a qualitative assessment is all that is required to adequately adjust the gastric restriction device. Knowing the volume of the test substance ingested and the time it takes for that volume of material to completely pass the stomal opening allows an accurate determination of average flow rate.

Conveniently, the circuitry provided can be self-calibrating, such that shortly after powering up the sensor and associated circuitry, the device would establish a base line capacitance value from which comparisons would then be made in the course of a flow rate testing procedure. It is also possible to provide a function as part of the overall apparatus that allows the operator to “zero” the instrument prior to performing a flow test, again for improved sensitivity.

Integral sensors suitable for use are not limited only to those capable of detecting changes in capacitance. Other means of sensing the flow of a fluid from the upper pouch to the lower pouch that detect other physical parameters may also be used successfully. For example, embodiments that use a sensor capable of detecting temperature, light, pH, magnetism, or a miniaturized radio frequency transmitting device in the form of a pill or capsule are also contemplated. Thus, as used herein, the terms “acoustic pill” and “acoustic capsule” refer to the same type of item.

Sensing temperature differentials could include the use of a sensor comprising a polyimide (kapton) substrate with an array of chip thermistors arranged in a linear fashion. Conveniently, this could then be covered by another layer of polyimide for protection. A set of circuit traces would also be on the polyimide substrate to connect up to each of the thermistors. This sensor assembly would then be adhered to the inside surface of the restriction device such that it would be in close contact with the tissue at or near the stomal opening.

Because the restriction device's inner surface is in intimate contact with the stomach tissue, a reference thermistor on the assembly would be effective to establish a baseline temperature. In using this type of monitor, the test substance is most conveniently a fluid having a temperature sufficiently different from normal body temperature, such that the fluid's presence at the stomal opening would be detected by the thermal sensor as an increased or decreased temperature relative to the temperature of the surrounding tissue of the gastric wall.

The precise temperature of the fluid ingested is not critical and it is expected that fluids over a wide range of temperatures would provide similar results in a flow condition or flow rate test. For safety and comfort, it might be desired to have the patient ingest a cooled fluid rather than a hot fluid, although either may be used. In addition, the difference between the tolerable low temperature ingested fluid and body temperature is significantly larger than the difference between the tolerable high temperature ingested fluid and body temperature, thus the measurable heat transfer can be greater when using a chilled fluid.

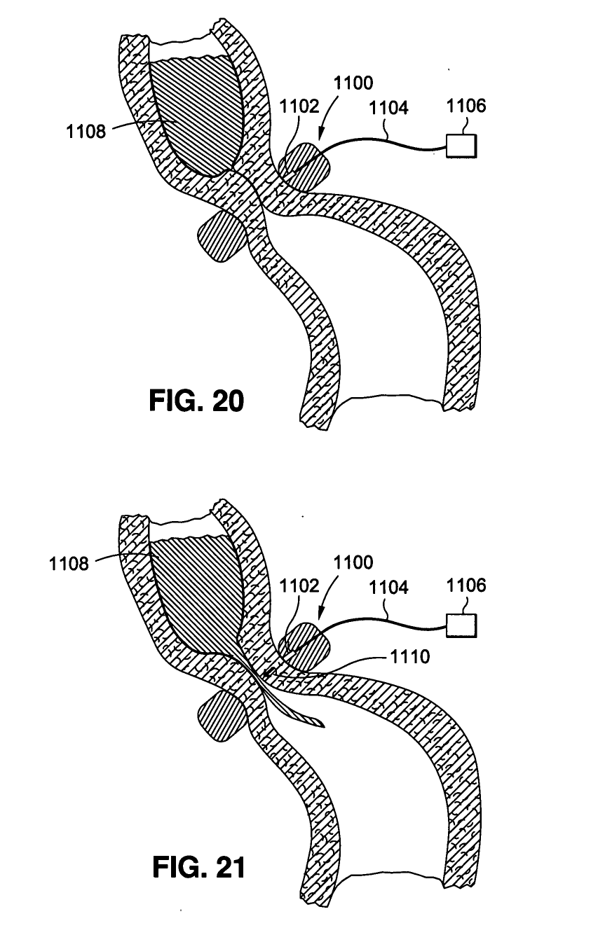

FIGS. 20 and 21 illustrate a gastric restriction device 1100 with a thermal sensor 1102. The thermal sensor can be a thermocouple, thermistor, RTD, optical temperature sensor, infrared detector or circuit with a temperature sensitive resistor. The resulting signal from the thermal sensor 1002 is carried by a conductor 1004 to a processing unit 1106, which can include a filter or amplifier to condition the signal. The processing unit can comprise a microprocessor. In some embodiments, the thermal sensor 1102 is embedded within the closing latch of the gastric restriction device. The sensitive portion of the thermal sensor 1102 is covered with a thin layer of thermally conductive but electrically insulative adhesive or epoxy.

In FIG. 20, test substance 1108 is ingested. The test substance 1108 is at a temperature which is different from that of body temperature. For example, the test substance is pre-cooled to 15° C. A gastric restriction device 1100 can be adjusted so that the test substance 1108 begins to flow past the stoma, as shown in FIG. 21. Heat flux 1110 (heat flowing from the stomach wall at the stoma to the test substance) results in a measurable drop in the temperature at the thermal sensor 1102, and a timer can be started. When the test substance passes completely, the surrounding body tissue will re-warm back to body temperature.

FIG. 12C provides one hypothetical depiction of temperature data from a thermal sensor, where the patient has ingested a test substance with a temperature greater than body temperature. In some embodiments a test substance with a temperature below body temperature can ingested. A baseline temperature 400 is measured before the start of the flow rate test. Typically, the baseline temperature will be about 37° C., which is normal core body temperature. As the sensor may also provide for a “zeroing” circuitry such that an averaged baseline temperature is set equal to zero, the data collected during the course of a flow test can be reported as a temperature difference 435 above or below a baseline temperature, as shown in FIG. 12C. The data may also be reported as a difference or as an absolute value. In addition, the reported data can either comprise an actual temperature sensed by the probe, or a difference between the sensed temperature and a previously determined or estimated baseline temperature.

Shortly after the time of ingestion of the test substance 430, at time T₀, an increase in temperature sensed by the thermal probe, and caused by the arrival of the test substance near the location of the thermal sensor positioned near the stomal opening, is detected. In the illustrated embodiment, the temperature difference 435 increases then decreases as the entire volume of fluid moves past the thermal sensor, finally returning to baseline at a later time, T₁, as the volume of material has completely passed through the stomal opening into the lower stomach pouch. At some point during the test, the difference between the sensed temperature and baseline temperature will be greater (in absolute value) than a pre-determined threshold. The time when the temperature differential rises above the threshold, until it falls under the threshold, will define a time interval 440. The interval between T₁ and T₀ will be the time taken for substantially the entire volume of fluid ingested to pass through the stomal opening. From this interval, and knowing the volume of material initially ingested, an average flow rate can thus be calculated as:

Flow rate(mL/sec)=Volume(mL)÷(T₁−T₀)(sec)

Similar data might be expected when measuring any physical property of an ingested substance, and so these data can be broadly viewed as illustrative of the expected results derived from any sensor system useable in accordance with the present disclosure.

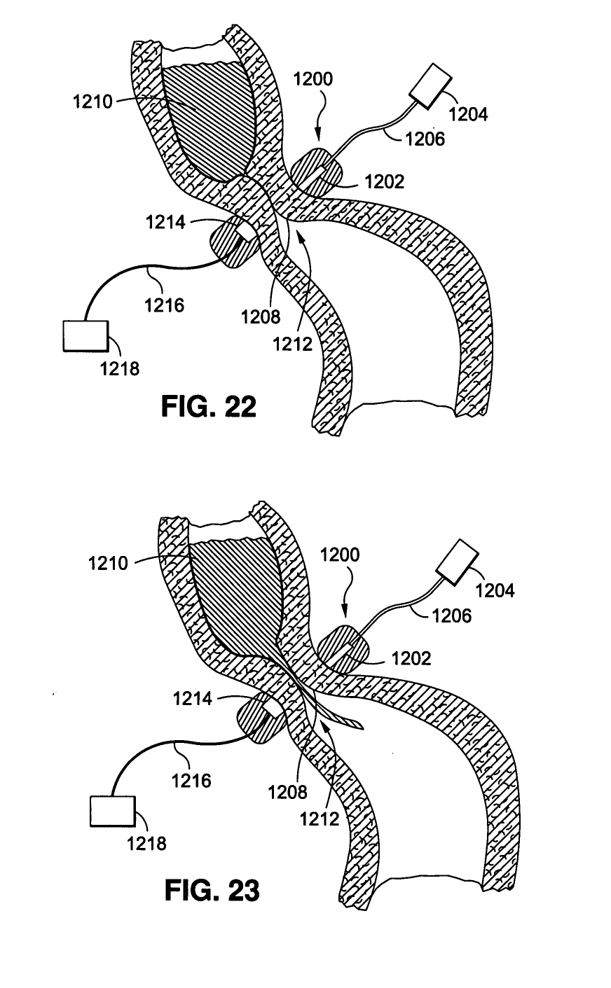

Embodiments using a light sensor are illustrated in FIGS. 22 and 23. A gastric restriction device 1200 comprises a fiber optic element 1202. A light source 1204 supplies light via one or more optical fiber 1206 to the fiber optic element 1202. In some embodiments, the fiber optic element 1202 comprises the polished end of the one or more optical fiber 1206. In some embodiments, the device can be configured such that the light is transmitted through the stomach wall 1208 at or near the stomal area 1212, with the light impinging on an photosensor 1214 located at the opposite side of the gastric restriction device 1200. A signal is created, which travels through signal line 1216 to a processor 1218. When a signal indicting that the light is sensed by the photosensor 1214 is received at the processor 1218, a no flow condition is indicated, as shown in FIG. 22. In some embodiments, the patient ingests an opaque test fluid 1210, for example coffee with cream. The gastric restriction device 1200 is adjusted until flow begins, as shown in FIG. 23. When a sufficient amount of the opaque test fluid 1210 passes between the fiber optic element 1202 and the photosensor 1214, light is prevented from falling on the photosensor, and no signal is received at the processor 1218, indicating a flow condition.

Optionally, the fiber optic element 1202 and the photosensor 1214 are both located on the same side of the gastric restriction device 1200, for example, next to each other. Instead of an opaque test fluid, a reflective test fluid, for example, a fluid that reflects infrared light, is ingested. In this case, flow of a reflective test substance results in light falling on the photosensor, while the absence of a reflective test substance results in little or no light impinges the photosensor. Thus, in this embodiment, a signal is indicative of a flow condition, while the absence of a signal is correlated with a no-flow condition.

Where the sensor was capable of detecting pH, it would likely be most accurate if the sensor was in direct contact with the luminal contents of the stomal opening. While this would require the probe to penetrate the gastric wall, micro-scale implantable electrodes capable of recording pH in vivo have been developed (see, for example, Johnson et al., Wireless Integrated Microsystems Engineering Research Center Annual Report 2005, the entirety of which is incorporated herein by reference).

In some embodiments, flow rate measurement may be determined using an electromagnetic sensor, and a conductive or magnetic fluid. The sensor design can comprise an inductive coil pattern on a polyimide substrate with a polyimide cover over the coil traces. In some embodiments, the sensor can be adhered to the inside surface of the restrictive device. In some embodiments, the sensor can comprise one or more wound coils embedded or housed on or within a closing latch portion of the restrictive device. The location of the sensor can be chosen to provide proximity to the stomal opening in order to provide effective detection of the conductive or magnetic fluid.

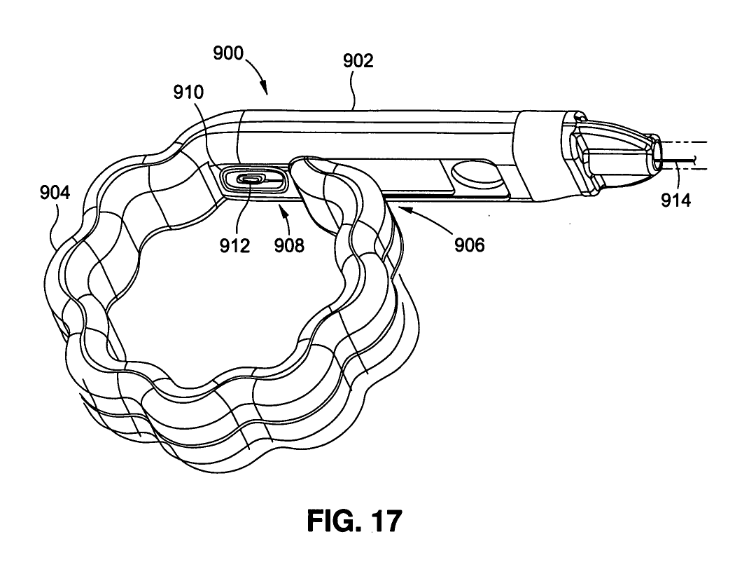

FIG. 17 illustrates a restriction device 900 comprising a non-dynamic portion 902 and a dynamically adjustable portion 904. In the illustrated embodiment, the non-dynamic portion 902 includes a latching mechanism 906. A sensor 908, comprising a transmitter coil 910 and a receiver coil 912, is located on the non-dynamic portion 902. Conductor wires 914 allow the passage of current to and from each of the coils. An alternating current is run through the transmitted coil 910 resulting in a changing magnetic field. The presence of a conductive or magnetic fluid alters the magnetic field, the field is sensed by the receiver coil 912 as a corresponding current is induced in it. This current is proportional to the amount of fluid sensed. The tissue of the constricted stomach wall is non-magnetic and thus does not affect the signal. The signal can be correlated to indicate the volume of the fluid present in the upper pouch. As this volume decreases (due to flow through the stomach, and thus, away from the upper pouch), a flow rate can be determined, based on the loss of volume per unit time.

A patient can be given a specific amount of the conductive or magnetic fluid to drink. The conductive or magnetic fluid can be made up of a small concentration of a biocompatible ferrous material mixed with a carrier of flavored water or other fluid. For example, magnetite (super-paramagnetic iron oxide) particles having a size range from 5 nm to 10 μm can be used. In some embodiments, particles size can range from about 500 nm to about 5 μm. A surfactant, such as oleic acid or silicone, can be used to coat the particles to improve their wettability and suspendability. A fluid, such as olive oil or low-calorie olive oil, can contain some oleic acid, improving the suspension of the coated particles within the oil.

As the fluid passes from the upper stomach pouch through the stomal opening to the lower stomach pouch, the presence of the conductive or magnetic fluid would be sensed by the inductive coil sensor. The sensor would in turn produce an output signal in response, this output signal being directly correlated to the presence of the conductive or magnetic fluid in the stomal opening. Alternatively, magnetite particles can be coated with silicone, and suspended in an aqueous solution, including, if desired, a flavorant. In some embodiments, a conductive fluid, such as gallium, may be used.

The system described is advantageous because the physician or other skilled technician is able to use the inductive coil sensor to determine the actual or real time flow rate of fluid through a restricted stoma. Some methods have been unable to discern the real time flow rates that occur through the restricted stoma. Not even barium consumption in combination with X-ray fluoroscopy can provide real-time feedback because there is no known way to visually quantify, with accuracy, a partially passed volume of barium through the restricted stoma. Physicians and others are interested in obtaining real time flow rate data because it more accurately reflects the behavior of fluid passing through the restricted stoma.

Fluid or food does not typically pass through the stoma at a steady rate. Peristaltic contractions typically cause an intermittent or periodic flow rate reading if assessing the flow rate in real time. The peak flow rate during this period can be an indicator of the effect of a tight restriction. For example, the likelihood of esophageal dilatation may be predicted by determining the peak flow rate. In addition to the peak flow rate, the frequency or consistency of the peristaltic contractions (i.e., the number of contractions per time) can also be determined. By identifying typical patterns of test flow traces, patients can be grouped by severity of esophageal condition or by peristaltic pattern, to help determine not only how tightly their restriction should be adjusted, but also, for example, whether a more conservative diet should be selected.

In addition, the peristaltic phenomenon can be used in conjunction with the real time flow measurement. For example, in some embodiments of a method of dynamic adjustment, the restriction device is tightened completely, causing complete occlusion at the stoma. Then the restriction device is slowly loosened until the desired stoma size is reached. By assessing a group of several peristaltic pulses, different degrees of stoma tightness can be more easily compared, without the need to ingest a large amount of test fluid.

As before, the output could be linked to a timing circuit such that the detection of the conductive or magnetic fluid would start a timer as the fluid was first present in the stomal opening, and stop the timer after the fluid had completely passed through the opening into the lower stomach pouch. Threshold values could also be established in order to more accurately control the start and stop of the timer. Once a time interval has been determined, the flow rate can be calculated by the same method as described above for thermal sensing systems.

As described above for sound and Doppler ultrasound detection, it may at times be sufficient to determine simply a flow-versus-no-flow condition in order to adjust the gastric restriction device. Any of the embodiments described above, and obvious variants that may be resorted to, are equally effective in providing a flow or no flow indication to a user.

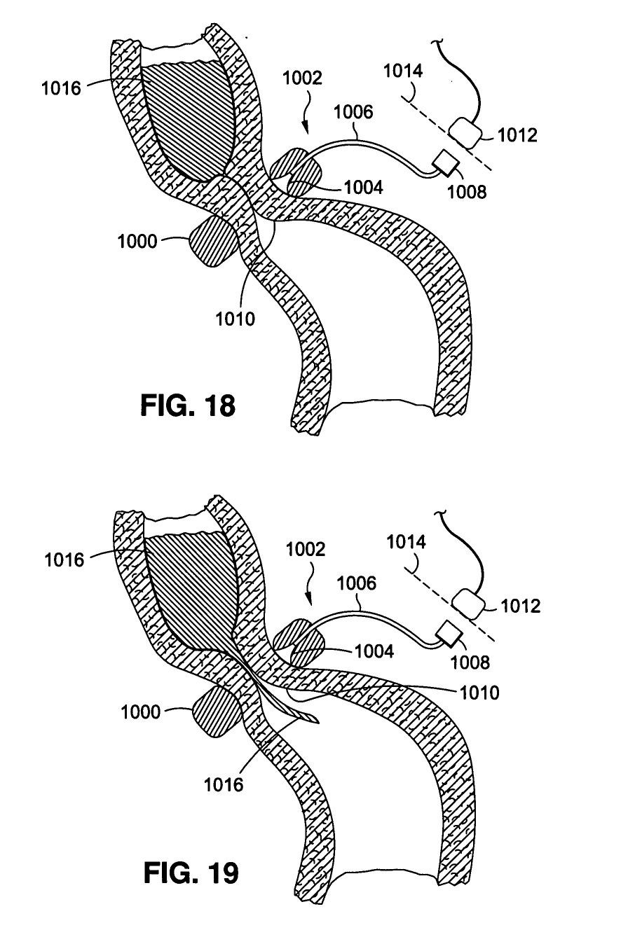

Some embodiments for a sound sensing restriction device that can be used with a sound producing fluid is illustrated in FIG. 18 and FIG. 19. A gastric restriction device 1000 comprising a mini-stethoscope 1002 is illustrated and comprises a head 1004, elongated sound pipe 1006 and implantable interface 1008. The head 1004 and implantable interface 1008 may optionally be covered with a vibrating membrane. When fluid is in a dynamic state, as for example, when flowing through the stoma, resulting sound waves are conducted through the stomach wall 1010, through the orifices of the head 1004 and the sound pipe 1006, eventually reaching the interface 1008. The interface 1008 can include a sound resonator to amplify the sound, analogous to a megaphone.

An external listening device 1012 senses the sound waves that pass from the interface 1008 and through the fat and skin 1014. In the no-flow condition, as illustrated in FIG. 18, no significant sound is detected by the system. In the flowing state, illustrated in FIG. 19, the sound of the test substance 1016 (e.g., fluid) passing the stoma is detected by the system. To increase the amplitude of the sonic signal, a sound producing fluid, such as that described in FIG. 7, can be used. The external listening device 1012 can comprise a stethoscope, an electric stethoscope, a microphone or a pickup, or any other sensor of sound known to those of skill in the art.

The sound pipe 1006 can include additional materials to conduct the sound, such as, for example, an internal metallic coil or stainless steel, which is minimally restrained so that it can vibrate within a pre-determined frequency range. The external listening device 1012 can be tuned or the signal can be filtered so that only a specific range of frequencies are received at maximal intensity. The head 1004 may consist of a funnel shaped cavity located inside the closing latch of the gastric restriction device and can be molded, machined or formed by another method.

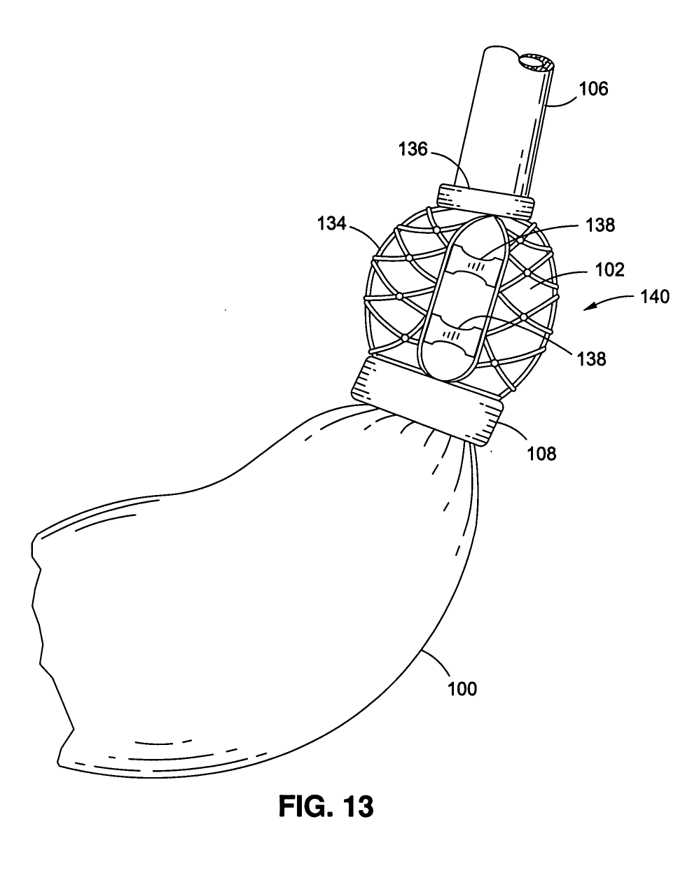

FIG. 13 illustrates embodiments for a gastric restriction device further comprising a slippage monitor. As described above, slippage of gastric restriction devices can occur, and can result in reduced effectiveness of the device due to expansion of the upper stomach pouch beyond a desirable size. Detecting movement of the gastric restriction device from an initial placement position to an undesired position can be readily determined with the disclosed system. A slippage monitor 140 comprises, in some embodiments, an upper securement portion 136, a mesh 134, and stress/strain sensors 138. The gastric restriction device 108 is placed, as in some other embodiments, for example, laparoscopically. The mesh 134, such as, for example, a sock or sleeve, is placed over the upper stomach pouch 102 formed by the device.

The stress/strain sensors 138 will detect any change in shape or size of the upper stomach pouch 102, as may occur when the gastric restriction device 108 slips. The sensors 138 could be calibrated to account for normal shape and size changes unrelated to slippage but rather which are due to normal stomach movement. As with the other sensors, the stress/strain sensors 138 would output a signal to a telemetry unit 155 that would relay data from the sensors to an external receiver 500. The telemetry unit 155 may be adjacent to the slippage monitor 140 or may be located at another convenient location in the body.

FIG. 11B additionally depicts the general arrangement of an internally mounted sensor that further includes erosion sensing electrodes 132. These electrodes would allow measurement of ionic impedance, for example. Should the band erode the stomach wall and contact the interior of the stomach, the stomach contents will interact with the electrodes and a change in impedance will be detected. Erosion sensors could include one or more pH sensors to take advantage of the low pH conditions in the interior of the stomach (typically in the range of pH 1-2) to indicate when erosion through the stomach wall has occurred.

In order to detect erosion even earlier, when the band has not yet eroded through the entire stomach wall, one or more temperature (thermal) sensors or oxygen sensors may be used instead of the one or more pH sensors. Temperature sensors can include thermocouples, thermistors, RTD, or optical fiber temperature sensors. The temperature sensors can sense erosion by more than one method. First, because erosion can stem from an infection, local inflammation can be quantified by one or more temperature sensors located on the band. The sensors may be located around the inner surface of the band or the outer surface or even side of the band. One of the locations providing a nidus for band erosions is the anterior suturing of the stomach wall around the band (in order to minimize anterior slippage).

A first temperature sensor located at the portion of the band that is near this site (for example, a point on the outer diameter of the band) can sense a rise in temperature, for example 2° C., that can be correlated with a localized inflammatory response. A second temperature sensor located away from the implanted portion of the patient senses normal body temperature, helping to differentiate between local inflammation and a systemic febrile condition. In some embodiments, the temperature sensor can be used to sense the thinning of the stomach wall that occurs as a band erodes through the external to internal layers, serosa, muscularis externa, submucosa, and mucosa, respectively. If a band is partially eroded, then a colder than body temperature test substance or a warmer than body temperature test substance will result in a greater change in the sensed temperature of a temperature sensor located on the inner surface of the band, due to the shorter distance of heat conduction through the now thinner, eroded stomach wall.

In some embodiments, one or more oxygen sensors may be used in place of the other sensors mentioned in order to actively monitor ischemia. Ischemia of the blood vessels in the stomach wall is thought to be a precursor of some erosions. Types of oxygen sensors include oxygen saturation and oxygen tension sensors, including MEMS-based sensors.

The sensor embodiments described herein which require power may be powered by a power source 406, for example an internal battery. They may also be powered using inductive coupling, either directly, or via an implanted capacitor which is charged via inductive coupling. Sensors may thus be operated continuously or may be powered on and off as desired. Alternatively, energy harvesting may be used in order to supply power to the sensors, or for that matter, for the adjustment of the gastric restriction device. The types of energy that may be harvested include, without limitation, solar, thermal, vibrational, inertial, gravitational, and radiowave. Energy harvesting can be performed by nanogenerators, such as for example, an array of aligned nanowires grown on a substrate.

The various sensor embodiments described herein can have a telemetry unit 155 that provides a means for relaying data from the sensor 150 to a device that is capable of producing an audible or graphic output, or is capable of storing the data, such as a software program running on a computer. In the case of an integral sensor, data could be relayed either by wired leads provided as part of the implant or by wireless transmission means, such as radio transmitters designed for internal use. In this context, a sensor can be taken to mean, without limitation, any device that produces an output signal that is indicative of the flow condition through the stomal opening produced by the gastric restriction device. Thus, the sensor can be any one of the embodiments described above or any variants that those skilled in the art would contemplate in order to provide an internal sensor capable of detecting flow through the stomal opening.

FIG. 14 provides a block diagram of one possible arrangement of an integral sensor 150, telemetry unit 155, external processor 504 and display 502. A sensor 150 provides an output signal to a telemetry unit 155. In some embodiments (not shown), the telemetry unit 155 comprises a transceiver 400, and the output of the sensor 150 would pass directly to the transceiver 400 for transmission to an external receiver 500. In some embodiments, the telemetry unit 155 may further include an optional telemetry unit processor 402.

As shown in FIG. 14, the telemetry unit processor 402 receives the output signal from the sensor 150. The telemetry unit processor 402 may include optional circuitry for noise suppression, a timer mechanism, or may be programmed to signal the transceiver 400 when the output signal from the sensor 150 is above a certain pre-determined threshold. The telemetry unit 155 may also include telemetry unit memory 404 operative to either store data from the telemetry unit processor 402, or which could be programmed with data useable by the telemetry unit processor 402 in processing a message for the transceiver 400 to relay to the external receiver 500.

Some embodiments include an external receiver 500, which receives a signal from the transceiver 400. The signal can comprise data from the sensor 150, timing information from the telemetry unit processor 402, and other types of information those skilled in the art would consider as conventional messages between two devices. In some embodiments, the data will be sent in digital form, and will include conventional forms of error correction and checks on data integrity. It is also possible to send information via analog modes. Transmission can be by any form of electromagnetic energy and wavelength suitable for the transmission of data.

The external receiver 500 can be optionally configured to send and receive data to and from the telemetry unit 155. There may also be included an external processor 504. The external processor 504 will receive signals from the receiver 500 corresponding to signals generated by the telemetry unit 155. The external processor 504 can provide an output to a display 502. There can also be included an external memory 506 and a user interface 508.

The display 502 can be a graphical display of acoustic spectral information, a data output value from the processor, or an indicator lighting system to tell the person performing the flow test when the flow rate is within a desired range or when a flow or no flow condition is detected. The graphic interface can be used to program the external processor or to input patient data, for example. In its simplest form, the display 502 can provide an indicator (e.g., audible or visible) to direct the user to start or stop a manual timing device in response to the property sensed (e.g., temperature, pH, capacitance) being above or below a certain pre-determined threshold. Alternatively, a display such as a tone or a light indicating means such as an LED or an array of LEDs might be used to indicate the presence or absence of flow.

Thus, there may also be provided, in some embodiments, a method of adjusting a gastric restriction device where the device is first adjusted to close off the stomal opening, as illustrated in FIG. 3. The patient then ingests a small volume of a test substance while the gastric restriction device is gradually opened in order to create a stomal opening that just permits flow. When flow begins to occur, the internal sensor 150 would detect the flow, a signal would be generated by the sensor 150 and telemetry unit 155, and the receiver 500 would detect the signal. The receiver 500 either directly, or via the processor 504, would cause an indication to appear on the display 502, indicating the fact that there was flow from the upper stomach pouch to the lower stomach pouch.

In more sophisticated embodiments, the display may provide a numerical readout from the computer processor of the result of flow duration, a flow rate calculation, for example calibrated in mL per second, or some other useful measure. Alternatively, the processor and display may be programmed such that when there is no flow a red LED is illuminated, where there is detectable flow or flow is within a desired range a green LED is illuminated, and if flow is greater than a desired range, a yellow LED is illuminated (the choice of color being purely discretionary). The display options may be even simpler in that a red LED is illuminated when there is no flow and a green LED illuminates when flow is detected. Various combinations of visual displays are possible, and thus the choice of display is not meant to limit the scope of the embodiments disclosed. In some embodiments, a combination of an audible and visible display are provided. Thus, in some embodiments, an alert such as a chime or some other kind of alert tone would be generated when flow was detected by the internal sensor. Tactile alerts such as vibration and temperature could also be used, alone or in combination, with the alerts described above.

The external memory 506 can be used to store data received from the internal sensor or to store programming parameters with which to calibrate the function of the system. For example, it could be useful for a patient to take weekly readings of such parameters as flow rate and then provide the data to a physician during the course of a regularly scheduled office visit. The telemetry unit 155, telemetry unit processor 402 and internal memory 404 could also be configured to store data from a series of test sessions and then be interrogated in order to download the data from the telemetry unit 155 as desired, for example, during a routine visit to a physician. In some embodiments, the band can be adjustable telemetrically, such that a physician could listen for an alert tone related to flow condition and then send a signal (e.g., telephonic, wireless, Internet, RF transmission, etc.) that would be relayed via the system to cause an adjustment mechanism on the gastric band to vary the opening until a desired setting was achieved. Storing data either in the internal memory 404 or external memory 506 would also provide a convenient means for the patient to download data after a series of measurements performed at home and then transmit that data to their physician electronically via email or other convenient electronic data transfer means.

In some embodiments, the ability to do home monitoring provides a distinct advantage in reducing the overall cost of after-surgery care and monitoring, as well as helping keep the physician better informed of the patient's progress without the need to schedule time-consuming and costly office visits. Storage of data permits comparison studies enabling establishment standardized criteria with which to calculate flow rates or to detect changes in the functioning of the gastric restriction device over time. Comparison could also lead to earlier detection of trends that would suggest the onset of a problem with either the placement or function of the device that has not yet manifested as any overt symptom in the patient, allowing for pre-emptive adjustment of the device in order to maintain functionality.

An object of the present disclosure is to provide an accurate measure of flow rate through the stomal opening produced by a gastric restriction device. However, depending on the nature of the material being consumed (e.g., fluid or food), the flow rate may vary. For water, the desired flow rate ranges from about 1 mL to about 20 mL second. In contrast, a slightly more viscous solution such as a dilute BaSo₄ suspension in water may have a slower flow rate, depending on the amount of barium included in the suspension. Much more concentrated BaSo₄ suspensions are commercially available, for example E-Z-PAQUE®, and have viscosities many times greater than water over the typical flow rates encountered in clinical applications. Solutions with even higher viscosities will be expected to move even more slowly through the opening. For example, it is known that solid food may be blocked by a stomal opening where liquids like water will readily pass. Therefore, another object of the disclosure is to provide a means of measuring flow rates with solutions having varying viscosity in order to better model the behavior of the various foods or beverages that the patient might normally consume, and thus derive a desired flow rate.

This may be accomplished through the use of test substances of varying viscosity in order to mimic the flow rate of a variety of ingested materials. For example, water at 20° C. has a viscosity of about 1 cP. Solutions with varying amounts of sucrose present can have viscosities ranging from about 3 cP to about 3,000 cP. Vegetable juices can have viscosity values ranging from less than about 10 cP to greater than about 3,000 cP. Solid foods have even higher viscosity values, as high as about 1×10⁵ cP or even greater. Thus a low viscosity test substance might be one with a viscosity of less than about 10 cP, a medium viscosity test substance might be in the range from about 10 cP to about 10,000 cP, and a high viscosity substance might have a viscosity from about 10,000 cP and higher. In some embodiments, a fluid having a viscosity in the range of about 0.5 to about 2 cP can be used.

Thus, in terms of usefulness of the data obtained in testing flow condition or flow rates, it will be desirable within a test session to determine either flow condition or flow rates for substances of differing viscosity. Thus, it is possible to not only check for flow through the stomal opening, but to ensure that the opening can accommodate desired rates of flow over a range of substance viscosities typical of fluids and foods ingested by most people. For greater certainty regarding the function of the restriction device, low, medium and high viscosity test fluids may be tested in turn as part of a single testing session, and in this way, the most beneficial adjustment of the gastric restriction device may be made based on a desired flow condition or flow rate. As the test is relatively easy, non-invasive, and of relatively short duration, testing multiple fluids would not be particularly burdensome to the patient and would potentially provide the physician or other caretaker with the best possible information in regards to the functioning of the gastric restriction device in order to adjust the device to provide a desired flow rate or flow condition.

Water is useful as a test fluid, especially when testing highly constricted stomal openings, as water has a relatively low viscosity and thus will flow relatively unimpeded through a wide range of stomal opening sizes. Viscosity is also affected by the temperature of the material, such that as temperature increases viscosity typically decreases. For example, water has a viscosity of about 1 cP at 20° C., which decreases to about 0.69 cP at 37° C. Thus, it would be advantageous to provide a means of equilibrating the test fluid to a pre-determined value prior to ingesting in order to reduce test to test variability. For example, the test fluid could always be heated to a temperature close to body temperature (37° C.) in order to minimize changes in fluid viscosity that would occur as the fluid warms in the body upon ingestion.

It will be of particular advantage to provide a test in which variability of various test parameters is minimized. As discussed above, the volume, temperature and viscosity of the test substance are among the factors that will affect the data recovered from a flow rate test as practiced by embodiments of the present disclosure. In order to minimize variability inherent to the test method and maximize the accuracy of the test results, some embodiments provide a kit with test substances comprising standardized test solutions, instructions on how to perform the test to achieve maximal accuracy and reproducibility, and optionally a Doppler ultrasound instrument suitable for home or clinical use.

The kit may include a set of standard test solutions of pre-determined viscosity, for example, a low viscosity, medium viscosity, and high viscosity solution, to evaluate flow of different types of materials through the stomal opening. For further ease of use, the test fluids could be pre-packaged in a one-use form of a known volume of fluid. By using a pre-packaged solution, the patient would use the correct volume of solution without incurring a risk of measuring error. As it might be further advantageous to ingest different volumes of fluids depending on their viscosity in order to obtain the most accurate measure of flow rate, pre-packaging test fluids in kit form would provide a simple way in which to provide test fluids of varying viscosities that are also optimized for volume. The kit could further include a heating device to heat the solution packages to a pre-determined value, for example 37° C., which is the generally accepted normal human body temperature, to minimize any changes in viscosity that would occur upon ingesting a test solution. In some embodiments, the kits may further provide solutions of different viscosities for use at different times of the day. It is known that flow past gastric restrictions exhibit diurnal variation, and so ingesting a solution with a higher viscosity when testing later in the day may be more useful.

The test solutions could be further coded with a simple letter or number code (e.g., A, B, C or 1, 2, 3), and the coding could be used in conjunction with a calibration system on the Doppler instrument such that a correspondence algorithm would reference the solution code as pertaining to a particular volume and viscosity previously programmed or programmable into the processor. Coding would also minimize operator errors in terms of inputting volume or viscosity measures, values which would typically comprise multiple digits and whose input could be prone to operator error.

Use of a software interface would also permit display of the sound files in a graphic format that permits a simple determination of fluid transit time in the stomach by measuring the time interval during which the sound intensity is greater than a pre-determined threshold. Dividing the volume of fluid ingested by the transit time would thus provide a direct measure of flow rate past the gastric restriction device. Accordingly, based on the data collected, a physician using standardized criteria that permit an accurate calculation of flow rate could adjust the gastric restriction device to provide precise adjustment of the restriction device to either increase or decrease flow as required.

In addition to the adjustment of the gastric restriction device, a feature can be included on the gastric restriction device that allows for automatic adjustment to counteract the diurnal variation in the condition of the stomach wall at the stoma. For example, a gastric restriction device with an integral dynamic actuation system (for example, using an implanted motor), can increase the diameter of the device by about 0.1 mm to about 0.5 mm every morning, and then decrease the diameter by the same amount prior to lunch time. With this feature, the restriction in the device will be similar at breakfast, lunch and dinner. The specific times of adjustment can be programmed into the device, depending on the work or sleep schedule of the patient.

In some embodiments, this automatic adjustment can be coupled to sensing information sensed by a flow sensor coupled to the gastric restriction device. For example, the first attempted swallow in a new day could be the trigger for the automatic increase in diameter (by about 0.1 mm to about 0.5 mm). In some embodiments, the patient does not have the ability to adjust the gastric restriction device to any diameter but can adjust the gastric restriction device to a pre-determined “morning” setting and an “afternoon” setting.

A patient can also have an implanted radio frequency identification device (RFID), which can be read from or written to using a processor included as part of a telemetry unit 155. The RFID could be used to store a variety of pieces of data including, but not limited to, personal patient information or information regarding adjustment of the gastric restriction device, or a patient's weight, for example, or trends showing success or lack thereof in the weight loss program. The RFID can also be used for security purposes, for example, for determining which model of device the patient has implanted, assuring that the correct data, codes, and algorithms are used in connection with interrogating or programming the device. In addition, the RFID can assure that a device, for example a device made by another manufacturer or one that is not appropriately calibrated, qualified or licensed, cannot be used with a particular receiver or programming module.

The skilled artisan will recognize the interchangeability of various features from different embodiments. Similarly, the various features and steps discussed above, as well as other known equivalents for each such feature or step, can be mixed and matched by one of ordinary skill in this art to perform compositions or methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

What is claimed is:

1. A system for use in controlling food intake in a patient, the system comprising:
a gastric restriction device that engages the patient's stomach between proximal and distal gastric regions that are connected by a stomal opening; and
a sensor coupled to the gastric restriction device;
wherein the sensor is configured to reside within the patient's body when the gastric restriction device is engaged with the stomach; and
wherein the sensor outputs information indicative of at least one of:
a presence of a test substance within the stomach;
a movement of the test substance within the stomach;
a movement of the gastric restriction device from a first position to a second position; and
an erosion of a wall of the stomach.

2. The system of claim 1, wherein the information indicative of the movement of the test substance comprises at least one of a flow rate, a flow velocity, and an occurrence of flow.

3. The system of claim 1, wherein the sensor is electrically coupled to the gastric restriction device.

4. The system of claim 1, wherein the sensor is mechanically coupled to the gastric restriction device.

5. The system of claim 1, wherein the sensor is located in or on the gastric restriction device.

6. The system of claim 1, wherein the sensor comprises a thermal sensor that detects a temperature that is influenced by the test substance.

7. The system of claim 1, wherein the sensor comprises a magnetic field sensor that detects a magnetically detectable property of the test substance.

8. The system of claim 1, wherein the sensor comprises a radiofrequency receiver that detects an electromagnetic signal emitted by the test substance.

9. The system of claim 1, wherein the sensor comprises a capacitance sensor.

10. The system of claim 9, wherein the capacitance sensor detects a substance present in the stomal opening.

11. The system of claim 1, wherein the sensor comprises an impedance sensor.

12. The system of claim 11, wherein the impedance sensor detects an indication of the erosion of the wall of the stomach.

13. The system of claim 1, wherein the sensor comprises a pH sensor.

14. The system of claim 13, wherein the pH sensor detects at least one of a substance present in the stomal opening and an indication of the erosion of the wall of the stomach.

15. The system of claim 1, wherein the sensor comprises an oxygen sensor, effective to detect an indication of the erosion of the wall of the stomach.

16. The system of claim 1, wherein the sensor detects light.

17. The system of claim 1, wherein the sensor detects acoustic energy.

18. The system of claim 1, wherein the sensor is configured to detect Doppler shift echoes from ultrasound.

19. The system of claim 1, further comprising the test substance.

20. The system of claim 19, wherein the test substance comprises at least one of a food, a beverage, and a gastric secretion.

21. The system of claim 19, wherein the test substance comprises a fluid.

22. The system of claim 21, wherein the fluid has a viscosity between about 0.5 cP and about 2.0 cP at 20° C.

23. The system of claim 21, wherein the test substance comprises a scattering agent that increases an echogenicity of the fluid.

24. The system of claim 19, wherein the test substance emits or reflects acoustic energy.

25. The system of claim 24, wherein the acoustic energy comprises at least one of audible sound, ultrasound, and Doppler shift echoes.

26. The system of claim 24, wherein the test substance comprises an effervescent solution.

27. The system of claim 24, wherein the test substance comprises an acoustic capsule.

28. The system of claim 19, wherein the test substance comprises at least one of a light-reflecting material and a light-absorbing material.

29. The system of claim 1, further comprising a telemetry unit that relays an output signal from a portion of the system that is inside the patient's body to an external receiver located outside the patient's body.

30. The system of claim 29, wherein the telemetry unit further comprises a telemetry unit processor that processes a signal outputted from the sensor.

31. The system of claim 29, further comprising the external receiver.

32. The system of claim 1, further comprising a display, operative to indicate at least one of:
the presence of the test substance within the stomach;
the movement of the test substance within the stomach;
the movement of the gastric restriction device from the first position to the second position; and
the erosion of the wall of the stomach.

33. The system of claim 32, wherein the display provides an alert that is at least one of audible, visible, and tactile.

34. The system of claim 33, wherein the alert comprises at least one of an audible tone, an LED, a video display, a numerical display, vibration, and heat.

35. A method, of monitoring performance or placement of a gastric restriction device, comprising:
with a gastric restriction device, engaging a patient's stomach between proximal and distal gastric regions that are connected by a stomal opening;
with a sensor that is coupled to the gastric restriction device and that resides within the patient's body when the gastric restriction device is engaged with the stomach, sensing information indicative at least one of:
a presence of a test substance within the stomach;
a movement of a test substance within the stomach;
a movement of the gastric restriction device from a first position to a second position; and
a presence of an erosion of a wall of the stomach.

36. The method of claim 35, wherein the information indicative of the movement of a test substance comprises at least one of a flow rate, a flow velocity, and an occurrence of a flow.

37. The method of claim 35, further comprising transmitting data based on the sensed information, located inside the patient's body, to an external receiver located outside of the patient's body.

38. The method of claim 37, further comprising adjusting a size of the gastric restriction device based on the transmitted data.

39. The method of claim 38, further comprising adjusting the gastric restriction device to achieve a flow rate of the test substance through the stomal opening in a range between about 1 mL per second and about 20 mL per second.

40. The method of claim 38, further comprising adjusting the gastric restriction device to achieve a flow rate of the test substance through the stomal opening in a range from about 5 mL per second to about 15 mL per second.

41. The method of claim 35, wherein the test substance is equilibrated to a temperature about equal to a body temperature of the patient prior to administration of the test substance to the patient.

42. The method of claim 35, wherein the sensor is located in or on the gastric restriction device.

43. The method of claim 35, wherein the sensor comprises a thermal sensor, and the test substance has a temperature distinguishable from a body temperature of the patient.

44. The method of claim 35, wherein the sensor comprises a magnetic sensor, and the test substance has magnetically detectable properties.

45. The method of claim 35, wherein the sensor comprises a radiofrequency receiver, and the test substance comprises a radio transmitter.

46. The method of claim 35, wherein the sensor comprises a capacitance sensor.

47. The method of claim 46, wherein the capacitance sensor detects a substance present in the stomal opening.

48. The method of claim 35, wherein the sensor comprises an impedance sensor.

49. The method of claim 48, wherein the impedance sensor detects an indication of the erosion of the wall of the stomach.

50. The method of claim 35, wherein the sensor comprises a pH sensor.

51. The method of claim 50, wherein the pH sensor detects at least one of a substance present in the stomal opening and an indication of the erosion of the wall of the stomach.

52. The method of claim 35, wherein the sensor comprises an oxygen sensor, effective to detect an indication of the erosion of the wall of the stomach.

53. The method of claim 35, further comprising administering the test substance to the patient.

54. The method of claim 53, wherein the test substance comprises at least one of a food, a beverage, and a gastric secretion.

55. The method of claim 53, wherein the test substance comprises a fluid.

56. The method of claim 55, wherein the fluid has a viscosity between about 0.5 cP and about 2.0 cP at 20° C.

57. The method of claim 53, wherein the test substance emits or reflects acoustic energy.

58. The method of claim 57, wherein the acoustic energy comprises at least one of audible sound, ultrasound, and Doppler shift echoes.

59. The method of claim 57, wherein the test substance comprises an effervescent solution.

60. The method of claim 57, wherein the test substance comprises an acoustic capsule.

61. The method of claim 35, further comprising sensing acoustic energy with the sensor.

62. The method of claim 35, further comprising sensing light energy with the sensor.

63. The method of claim 35, wherein the test substance comprises at least one of a light-reflecting material and a light-absorbing material.

64. The method of claim 35, wherein the sensor is configured to detect Doppler shift echoes from ultrasound.

65. The method of claim 35, wherein the test substance comprises a scattering agent that increases an echogenicity of the fluid.

66. A system for use in controlling food intake in a patient, the system comprising:
means for engaging the patient's stomach between proximal and distal gastric regions that are connected by a stomal opening; and
means for sensing coupled to the means for engaging;
wherein the means for sensing is configured to reside within the patient's body when the means for engaging is engaged with the stomach; and
wherein the means for sensing outputs information indicative of at least one of a presence of a test substance within the stomach, a movement of a test substance within the stomach, a movement of the gastric restriction device from a first position to a second position, and a presence of an erosion of a wall of the stomach.
